(19) United States
(12) Patent Application Publication
(10) Pub. No.: US 2020/0331882 A1
(43) Pub. Date: Oct. 22, 2020

(54) COMPOUND FOR SIMULTANEOUSLY INHIBITING LSD1 AND HDAC TARGETS AND APPLICATION THEREOF

(71) Applicant: PEKING UNIVERSITY SHENZHEN GRADUATE SCHOOL, Shenzhen (CN)

(72) Inventors: Junmin QUAN, Shenzhen (CN); Qinkai LI, Shenzhen (CN); Xin ZENG, Shenzhen (CN); Zhengshuang XU, Shenzhen (CN); Hong YUE, Shenzhen (CN); Tingyao ZHANG, Shenzhen (CN); Jiamiao FU, Shenzhen (CN); Xinyi HUANG, Shenzhen (CN)

(73) Assignee: PEKING UNIVERSITY SHENZHEN GRADUATE SCHOOL, Shenzhen (CN)

(21) Appl. No.: 16/920,450

(22) Filed: Jul. 3, 2020

Related U.S. Application Data
(63) Continuation of application No. PCT/CN2018/071317, filed on Jan. 4, 2018.

(51) Int. Cl. C07D 401/04; C07D 241/04; C07D 265/30; C07D 233/61; C07D 211/34; A61P 35/00

(52) U.S. Cl. CPC C07D 401/04; C07D 241/04; C07D 265/30; A61K 45/06; C07D 211/34; A61P 35/00; C07D 233/61

(57) ABSTRACT

A compound having a general structural formula as shown in Formula I: X-AB-Y (Formula I); in the above Formula I, X is selected from any one of —CO2H, —CONHZ, —CH═CH—CO2H, —CH═CH—CONHZ, wherein Z is selected from any one of substituted or unsubstituted C1-C12 alkyl, substituted or unsubstituted aryl, and hydroxyl; Y=—NR1R2, wherein NR1R2 is a substituted or unsubstituted 3- to 9-membered nitrogen-containing heterocycloalkyl; A and B are each independently selected from substituted or unsubstituted phenylene, substituted or unsubstituted azaphenylene. The compound or corresponding pharmaceutical salt thereof can inhibit LSD1 and HDAC target proteins at the same time, thus inhibit the proliferation of many kinds of tumor cells and have good antitumor effect.

### CROSS-REFERENCE TO RELATED APPLICATION

This application is a continuation of PCT application No. PCT/CN2018/071317, filed on Jan. 4, 2018. The patent application is hereby incorporated by reference in its entirety.

### TECHNICAL FIELD

The present invention belongs to the technical field of medicines, and particularly relates to a compound for simultaneously inhibiting LSD1 and HDAC targets and an application thereof.

### BACKGROUND ART

Histone modifications include methylation, acetylation, phosphorylation, ubiquitination, and the like. Histone methylation occurs at arginine and lysine residues of histones H3 and H4, and can occur 1-2 times (arginine) or 1-3 times (lysine). It is regulated by histone methyltransferase (HMT) and histone demethylase (HDM) and characterized by transcriptional activation or gene silencing. Histone acetylation process is jointly regulated by histone acetyltransferase (HAT) and histone deacetylase (HDAC). Most histones in the transcriptional activation region show highly acetylated state, while deacetylated state usually shows gene silencing. Abnormal histone modification is also an important cause of cancer development.

The action mechanism of histone deacetylases (HDACs) is to remove the acetyl group of nucleosome histone lysine residue, reduce the space between nucleosome and DNA, at the same time, make histone return to positive electricity, enhance the electrostatic interaction between nucleosome and DNA, make DNA tightly entangled on nucleosome, thus hinder the binding of transcription factor to DNA and inhibit the process of transcription. Histone acetylation is closely related to the development of cancer and the expression of oncogenes and tumor suppressor genes. Many cancers show abnormal expression of histone acetyltransferase and deacetylase, usually characterized by overexpression or activation of HDACs, which inhibits the expression of specific genes. Studies have shown that core histone H4 in human cancer cells usually exhibits low acetylation at the overall level. Moreover, in addition to regulating the acetylation level of histone, HDACs can also bind and regulate many other protein factors, including transcription factors p53, E2F1, nuclear factor NF-κB, protein factor α-tubulin, Ku70, heat shock protein Hsp90, etc., and can target them for deacetylation to affect their activity. Studies have shown that histone deacetylation inhibitor (HDACi) can increase the level of histone acetylation in cancer cells, activate the expression of tumor suppressor genes and induce cancer cell apoptosis by inhibiting the effect of HDACs. Therefore, HDACs have become a hot spot in the field of cancer drug research and development. Like most anticancer drugs, HDACi can induce biological and morphological apoptosis in cancer cells. Animal experiments and clinical studies have shown that concentrations of HDACi that exhibit anti-cancer activity are less toxic to the host, dose-limiting toxicities generally include thrombocytopenia, nausea and fatigue, and in most cases these side effects are clinically controllable. HDACi can induce apoptosis by blocking cell cycle, and has anti-angiogenic, anti-spreading and immunoregulatory activities. HDACi may act directly on cancer cells to enhance their immunogenicity, as well as increase the activity of immune cells and promote cytokine production, thereby enhancing antitumor immunity.

Methylation/demethylation of histones is also an important research direction in epigenetics. With the discovery of the first histone lysine demethylase (LSD1), the view that histone methylation process is irreversible is revised, and laid the foundation for the discovery of more histone demethylases and function research thereof. The increase in the number of histone demethylases highlights the nature of dynamic regulation of histone methylation, which is a key chromatin modification involved in genome and gene regulation in eukaryotes. It plays an extensive role in cell proliferation, adipogenesis, spermatogenesis, chromosome segregation and embryonic development. Moreover, LSD1 can also promote tumor growth by inhibiting the activity of tumor suppressor p53. A series of studies have shown the specific biological effects of these enzymes and their potential links to human diseases.

LSD1 plays an indispensable role in the development and differentiation of mammals, which can regulate hormone levels, affect the proliferation and differentiation of hematopoietic cells and inhibit energy consumption and lipolysis. The overexpression of LSD1 is closely related to the occurrence and development of prostate cancer, breast cancer, colon cancer, neuroblastoma, non-small cell lung cancer and bladder cancer, and plays a significant role in the self-renewal and differentiation of stem cells and tumor stem cells. Inhibition of the expression or function of LSD1 has an important role in the treatment of cancer. LSD1, a new tumor drug target, has broad prospects for research, development and clinical application.

Histone demethylase LSD1 and histone deacetylase HDACs are key proteins of transcriptional suppressor complexes such as CoREST and NuRD. They extensively regulate the activation and inhibition of gene transcription by synergism, which are highly related to the occurrence and development of many kinds of tumors. Histone demethylase and histone deacetylase are highly expressed in a variety of tumor cells and have a variety of biological functions, including promoting tumor proliferation, promoting fat synthesis, inhibiting energy metabolism, inhibiting lipolysis, regulating cell differentiation and so on. Therefore, LSD1 and HDACs can be used as targets of antitumor drugs, and the research and discovery of their inhibitors are of great significance for the research and development of new antitumor drugs. At present, the main research on inhibitors is that single targeting LSD1 or HDACs, does not make full use of the synergistic effect of LSD1 and HDACs. It has been found in studies that the combination administration of LSD1 and HDACs small molecular inhibitors has a significant synergistic effect on the inhibitory activity of a variety of tumors, but the combination brings great challenges to clinical research because of the different pharmacokinetic properties of the combination drugs and the possible drug-drug interaction. Therefore, the study of bifunctional small molecule inhibitors targeting LSD1/HDACs at the same time has become a frontier research direction in this field.

### TECHNICAL PROBLEM

The present invention aims to overcome the defects in the prior art, provides a compound for simultaneously inhibiting LSD1 and HDAC targets and an application thereof, and aims to improve the synergistic effect of an existing epigenetic regulation drug in tumor treatment.

### TECHNICAL SOLUTION

In order to achieve the purpose, the present invention adopts the following technical solution: One aspect of the present invention provides a compound having a general structural formula shown in Formula I:

X-AB-Y Formula I

In the above Formula I,

X is selected from any one of —CO₂H, —CONHZ, —CH═CH—CO₂H, —CH═CH—CONHZ, wherein Z is selected from any one of substituted or unsubstituted C₁-C₁₂ alkyl, substituted or unsubstituted aryl, and hydroxyl;

Y=—NR₁R₂, wherein NR₁R₂ is a substituted or unsubstituted 3- to 9-membered nitrogen-containing heterocycloalkyl;

A and B are each independently selected from substituted or unsubstituted phenylene, substituted or unsubstituted azaphenylene.

Accordingly, a pharmaceutically acceptable salt of one of the above compounds of the present invention.

Accordingly, a pharmaceutical composition comprising a compound of the present invention as described above and a pharmaceutically acceptable carrier.

Finally, the present invention provides the use of a compound of the present invention as described above, or a pharmaceutically acceptable salt thereof in preparation of a drug for the treatment and/or prevention of a tumor or cancer associated with LSD1 and/or HDAC.

### ADVANTAGEOUS EFFECTS

A computer-aided design method is utilized in the present invention in combination with the experimental detection method, based on the crystal structure of the active pocket of LSD1 and HDAC1, to make full use of the common structure of the two kinds of protein active pocket to design and synthesize a series of biaryl small molecular compounds which can better inhibit LSD1 and HDACs at the same time. The compound or corresponding medicinal salt thereof can inhibit both LSD1 and HDAC target proteins, thus inhibiting the proliferation of tumor cells, showing a synergistic inhibitory effect in a variety of tumor cells, and can be used in the prevention and treatment of tumors or cancers associated with histone acetylation and methylation abnormalities.

### EMBODIMENTS OF THE INVENTION

In order to make the technical problems to be solved, the technical scheme and the beneficial effect of the present invention clearer, the present invention is further explained in detail in combination with the following embodiments. It is to be understood that the specific embodiments described herein are merely illustrative of the present invention and are not intended to be limiting thereof.

The compounds and derivatives thereof referred to in the embodiments of the present invention are named according to the IUPAC (International Union of Pure and Applied Chemistry) or CAS (Chemical Abstracts Service) nomenclature system. Thus, the compound groups specifically referred to in the embodiments of the present invention are illustrated and described as follows:

“Alkyl” means a straight or branched, monovalent, saturated aliphatic chain including, but not limited to, groups such as methyl, ethyl, propyl, isopropyl, butyl, isobutyl, pentyl, isopentyl, hexyl, and the like.

“Heteroalkyl” refers to a straight or branched, monovalent, saturated aliphatic chain linked to at least one heteroatom, for example, but not limited to, methylaminoethyl or other similar groups. “Alkenyl” means a straight or branched chain hydrocarbon having one or more double bonds and including, but not limited to, groups such as ethenyl, propenyl, and the like.

“Heteroalkenyl” means a straight or branched chain hydrocarbon having one or more double bonds linked to at least one heteroatom and including, but not limited to, groups such as vinylaminoethyl or the like.

“Alkynyl” refers to straight or branched chain hydrocarbons having one or more triple bonds and including, but not limited to, groups such as ethynyl, propynyl, and the like.

“Heteroalkynyl” means a straight or branched chain hydrocarbon having one or more triple bonds attached to at least one heteroatom and including, but not limited to, groups such as ethynyl, propynyl, and the like.

“Aryl” means a cyclic aromatic hydrocarbon including, but not limited to, groups such as phenyl, naphthyl, anthryl, phenanthryl, and the like.

“Heteroaryl” means a monocyclic or polycyclic or fused ring aromatic hydrocarbon in which one or more carbon atoms have been substituted with a heteroatom such as nitrogen, oxygen, or sulfur. If a heteroaryl contains more than one heteroatom, the heteroatoms may be the same or different. Heteroaryls include, but are not limited to, groups such as benzofuryl, benzothienyl, benzimidazolyl, benzoxazolyl, benzothiazolyl, benzopyranyl, furyl, imidazolyl, indazolyl, indolizinyl, indolyl, isobenzofuryl, isoindolyl, isoquinolinyl, isothiazolyl, isoxazolyl, naphthyridinyl, oxadiazolyl, oxazinyl, oxazoyl, phthalazinyl, pteridinyl, purinyl, pyranyl, pyrazinyl, pyrazolyl, pyridazinyl, pyrido[3,4-b]indolyl, pyridyl, pyrimidinyl, pyrrolyl, quinolizinyl, quinolinyl, quinoxalinyl, thiadiazolyl, thiatriazolyl, thiazolyl, thienyl, triazinyl, triazolyl, xanthenyl and the like.

“Cycloalkyl” refers to a saturated monocyclic or polycyclic alkyl group, possibly fused to an aromatic hydrocarbon group. Cycloalkyls include, but are not limited to, groups such as cyclopropyl, cyclobutyl, cyclopentyl, cyclohexyl, cycloheptyl, indanyl, tetrahydronaphthyl, and the like.

“Heterocycloalkyl” means a saturated monocyclic or polycyclic alkyl group which may be fused to an aromatic hydrocarbon group in which at least one carbon atom has been substituted by a heteroatom such as nitrogen, oxygen or sulfur. If a heterocycloalkyl contains more than one heteroatom, the heteroatoms may be the same or different. Heterocycloalkyl groups include, but are not limited to, groups such as azabicycloheptanyl, azetidinyl, indolinyl, morpholinyl, piperazinyl, piperidinyl, pyrrolidinyl, tetrahydrofuranyl, tetrahydroquinolinyl, tetrahydroindazolyl, tetrahydroindolyl, tetrahydroisoquinolinyl, tetrahydropyranyl, tetrahydroquinoxalinyl, tetrahydrothiopyranyl, thiazolidinyl, thiomorpholinyl, thioxanthenyl, thioxanyl, and the like.

“Cycloalkenyl” refers to an unsaturated, monocyclic or polycyclic alkenyl group having one or more double bonds, which may be fused to an aromatic hydrocarbon group, including, but not limited to, cycloethenyl, cyclopropenyl, or other similar groups.

“Heterocycloalkenyl” refers to an unsaturated, monocyclic or polycyclic alkenyl group having one or more double bonds, which may be fused to an aromatic hydrocarbon group, in which at least one carbon atom is substituted by a heteroatom such as nitrogen, oxygen or sulfur. If a heterocycloalkyl contains more than one heteroatom, the heteroatoms may be the same or different.

“Cycloalkynyl” refers to an unsaturated, monocyclic or polycyclic alkynyl group having one or more triple bonds, which may be fused to an aromatic hydrocarbon group, including, but not limited to, cycloethynyl, cyclopropynyl, or other similar groups.

“Heterocycloalkynyl” refers to an unsaturated, monocyclic or polycyclic alkynyl group having one or more triple bonds, which may be fused to an aromatic hydrocarbon group, in which at least one carbon atom is substituted by a heteroatom such as nitrogen, oxygen or sulfur. If a heterocycloalkyl contains more than one heteroatom, the heteroatoms may be the same or different.

It should be noted that in embodiments of the present invention, the symbol [symbol] represents the point of attachment of a moiety in a compound to the remainder of the compound.

In one aspect, embodiments of the present invention provide a compound having a general structural formula shown in Formula I:

X-AB-Y Formula I

In the above Formula I,

X is selected from any one of —CO₂H, —CONHZ, —CH═CH—CO₂H, —CH═CH—CONHZ, wherein Z is selected from any one of substituted or unsubstituted C₁-C₁₂ alkyl, substituted or unsubstituted aryl, and hydroxyl;

Y=—NR₁R₂, wherein NR₁R₂ is a substituted or unsubstituted 3- to 9-membered nitrogen-containing heterocycloalkyl;

A and B are each independently selected from substituted or unsubstituted phenylene, substituted or unsubstituted azaphenylene.

Further, in the above Formula I, A and B are each independently selected from: any one of

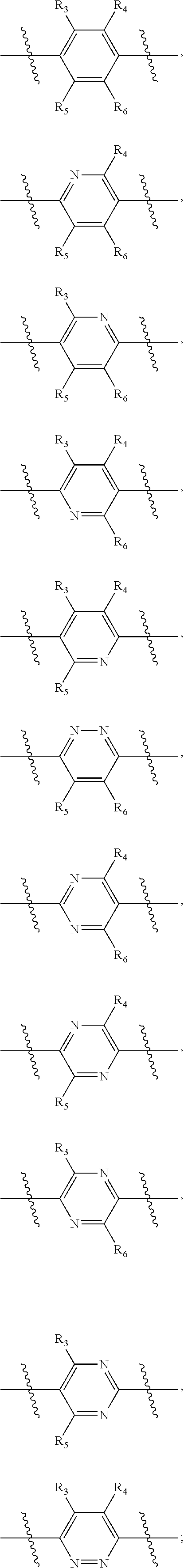

Wherein R₃, R₄, R₅ and R₆ are each independently selected from any one of substituted or unsubstituted C₁-C₁₂ alkyl, substituted or unsubstituted C₁-C₁₂ heteroalkyl, substituted or unsubstituted C₃-C₁₂ cycloalkyl, substituted or unsubstituted C₃-C₁₂ heterocycloalkyl, substituted or unsubstituted C₂-C₁₂ alkenyl, substituted or unsubstituted C₂-C₁₂ heteroalkenyl, substituted or unsubstituted C₃-C₁₂ cycloalkenyl, substituted or unsubstituted C₃-C₁₂ heterocycloalkenyl, substituted or unsubstituted C₂-C₁₂ alkynyl, substituted or unsubstituted C₂-C₁₂ heteroalkynyl, substituted or unsubstituted C₃-C₁₂ cycloalkynyl, substituted or unsubstituted C₃-C₁₂ heterocycloalkynyl, substituted or unsubstituted aryl, substituted or unsubstituted heteroaryl, aryl (C₁-C₁₂) alkyl, heteroaryl (C₁-C₁₂) alkyl, C₂-C₁₂ alkenyl (C₁-C₁₂) alkyl, C₂-C₁₂ alkynyl (C₁-C₁₂) alkyl, hydroxyl, amino, nitro, sulfo, halogen and hydrogen atom.

More preferably, R₃, R₄, R₅ and R₆ are each independently selected from any one of substituted or unsubstituted C₁-C₆ alkyl, substituted or unsubstituted C₁-C₆ heteroalkyl, substituted or unsubstituted C₃-C₆ cycloalkyl, substituted or unsubstituted C₃-C₆ heterocycloalkyl, substituted or unsubstituted C₂-C₆ alkenyl, substituted or unsubstituted C₂-C₆ heteroalkenyl, substituted or unsubstituted C₃-C₆ cycloalkenyl, substituted or unsubstituted C₃-C₆ heterocycloalkenyl, substituted or unsubstituted C₂-C₆ alkynyl, substituted or unsubstituted C₂-C₆ heteroalkynyl, substituted or unsubstituted C₃-C₆ cycloalkynyl, substituted or unsubstituted C₃-C₆ heterocycloalkynyl, substituted or unsubstituted aryl, substituted or unsubstituted heteroaryl, aryl (C₁-C₆) alkyl, heteroaryl (C₁-C₆) alkyl, C₂-C₆ alkenyl (C₁-C₆) alkyl, C₂-C₆ alkynyl (C₁-C₆) alkyl. More preferably, the R₃, R₄, R₅ and R₆ are each independently selected from substituted or unsubstituted C₁-C₄ alkyl.

Further, in the above Formula I, the structure of -AB— is selected from: any one of

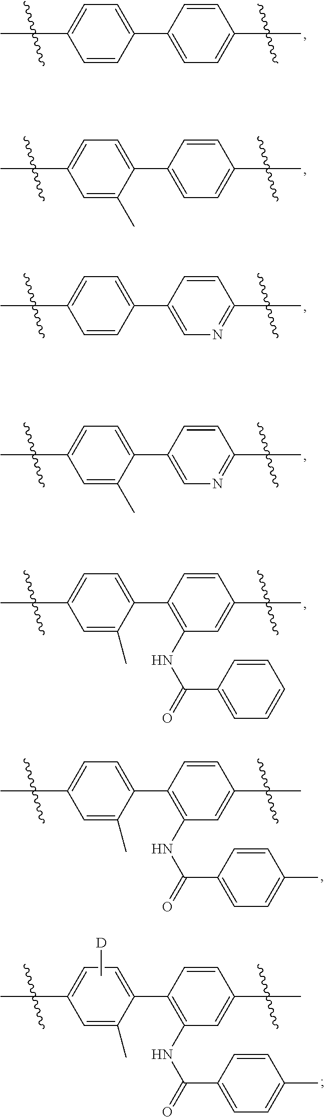

wherein D is halogen (e.g. fluorine, chlorine, bromine, iodine).

Further, in the above Formula I, Z is a substituted or unsubstituted C₁-C₄ alkyl group or a hydroxyl group.

Further, in the above Formula I, the structure of Z is

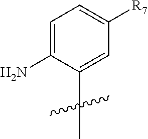

or hydroxy;

wherein R₇ is selected from any one of substituted or unsubstituted C₁-C₁₂ alkyl, substituted or unsubstituted C₁-C₁₂ heteroalkyl, substituted or unsubstituted C₃-C₁₂ cycloalkyl, substituted or unsubstituted C₃-C₁₂ heterocycloalkyl, substituted or unsubstituted C₂-C₁₂ alkenyl, substituted or unsubstituted C₂-C₁₂ heteroalkenyl, substituted or unsubstituted C₃-C₁₂ cycloalkenyl, substituted or unsubstituted C₃-C₁₂ heterocycloalkenyl, substituted or unsubstituted C₂-C₁₂ alkynyl, substituted or unsubstituted C₂-C₁₂ heteroalkynyl, substituted or unsubstituted C₃-C₁₂ cycloalkynyl, substituted or unsubstituted C₃-C₁₂ heterocycloalkynyl, substituted or unsubstituted aryl, substituted or unsubstituted heteroaryl, aryl (C₁-C₁₂) alkyl, heteroaryl (C₁-C₁₂) alkyl, C₂-C₁₂ alkenyl (C₁-C₁₂) alkyl, C₂-C₁₂ alkynyl (C₁-C₁₂) alkyl, hydroxyl, amino, nitro, sulfo, halogen and hydrogen atom.

Further, in the above Formula I, Y is a substituted or unsubstituted 5- or 6-membered nitrogen-containing heterocyclohydrocarbyl. More preferably, Y is selected from: any one of

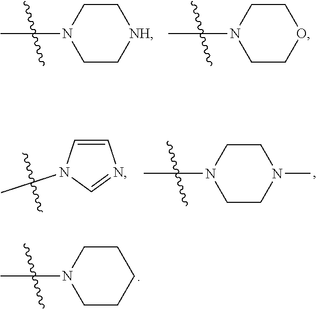

In particular, in a preferred embodiment of the present invention, the compound is selected from any one of

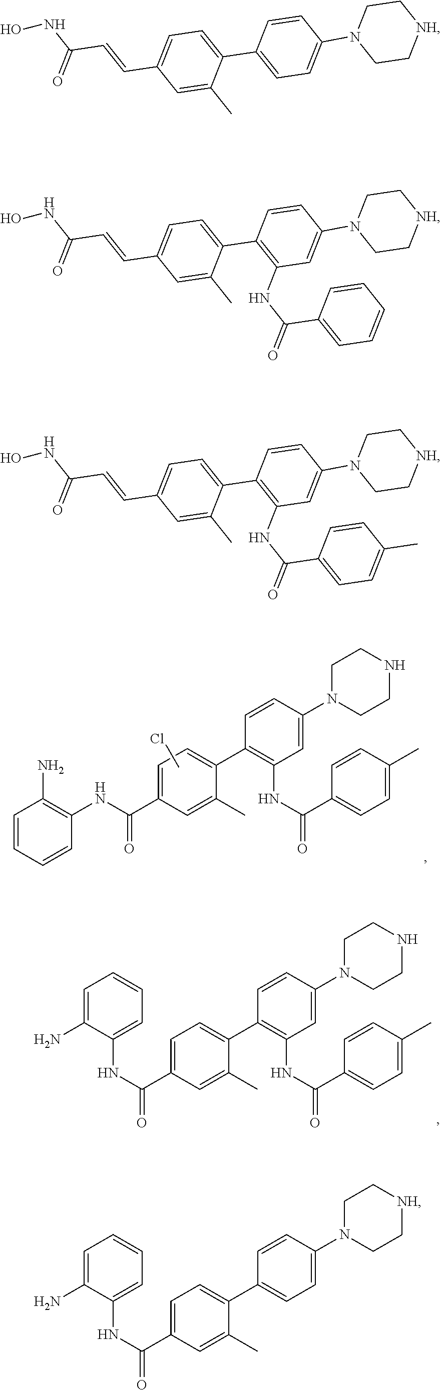

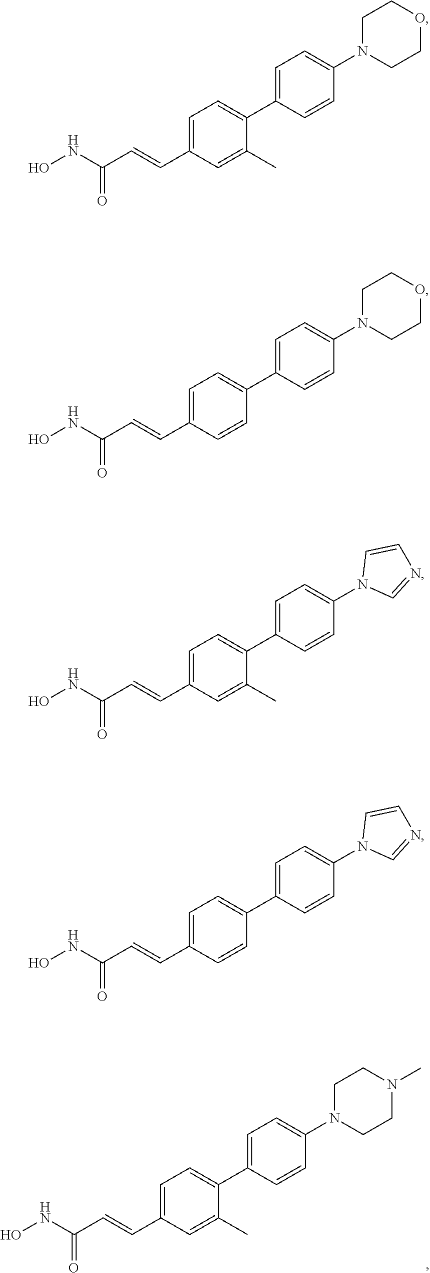 [SMILES: Cc1cc(/C=C/C(=O)NO)ccc1-c1ccc(N2CCOCC2)cc1 ; O=C(/C=C/c1ccc(-c2ccc(N3CCOCC3)cc2)cc1)NO ; Cc1cc(/C=C/C(=O)NO)ccc1-c1ccc(-n2ccnc2)cc1 ; O=C(/C=C/c1ccc(-c2ccc(-n3ccnc3)cc2)cc1)NO ; Cc1cc(/C=C/C(=O)NO)ccc1-c1ccc(N2CCN(C)CC2)cc1]

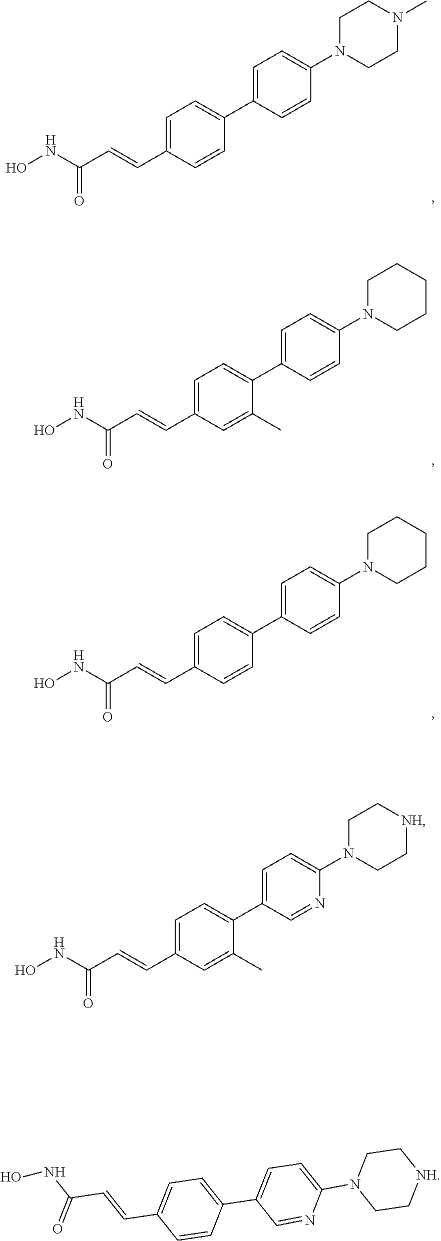 [SMILES: CN1CCN(c2ccc(-c3ccc(/C=C/C(=O)NO)cc3)cc2)CC1 ; Cc1cc(/C=C/C(=O)NO)ccc1-c1ccc(N2CCCCC2)cc1 ; O=C(/C=C/c1ccc(-c2ccc(N3CCCCC3)cc2)cc1)NO ; Cc1cc(/C=C/C(=O)NO)ccc1-c1ccc(N2CCNCC2)nc1 ; O=C(/C=C/c1ccc(-c2ccc(N3CCNCC3)nc2)cc1)NO]

In the embodiments of this specification, the preferred 16 compounds are designated as compounds of the ZZY series (i.e., ZZY-001 to ZZY-016 in turn). Accordingly, in an embodiment of the present invention, a pharmaceutically acceptable salt of the above compound of an embodiment of the present invention is also provided.

Accordingly, in an embodiment of the present invention, a pharmaceutical composition comprising a compound of the present invention as described above and a pharmaceutically acceptable carrier is also provided. In particular, auxiliary materials such as diluents, adhesives, absorbents, disintegrants, dispersants, wetting agents, co-solvents, buffers, surface activators, etc., needed in the preparation of pharmaceutical compositions are included. The pharmaceutical composition can be used for preventing and treating tumors or cancers related to histone acetylation and methylation abnormality. In particular, the pharmaceutical composition further comprises an anticancer drug. The anticancer drug is an LSD1 inhibitor and/or an HDAC inhibitor.

Finally, in an embodiment of the present invention, the use of a compound as described above in an embodiment of the present invention, or a pharmaceutically acceptable salt thereof in preparation of a drug for the treatment and/or prevention of a tumor or cancer associated with LSD1 and/or HDAC is also provided. In particular, the tumor or cancer is selected from: at least one of brain cancer, glioblastoma, leukemia, Bristlecone syndrome, Cowden's Disease, cerebellar dysplastic gangliocytoma, breast cancer, inflammatory breast cancer, Wilm's tumor, Ewing's sarcoma, rhabdomyosarcoma, ependymoma, medulloblastoma, colon cancer, head and neck cancer, kidney cancer, lung cancer, liver cancer, melanoma, kidney cancer, ovarian cancer, pancreatic cancer, prostate cancer, sarcoma, osteosarcoma, giant cell tumor of the bone and thyroid.

In embodiments of the present invention, the general formula for the synthesis of compounds of the ZZY series is shown below in the reverse synthesis analysis of ZZY-001. The synthesis of ZZY-001 can be carried out by HWE reaction of intermediates 1 and 2 to complete the carbon chain extension, wherein the hydroxamic acid at the carboxylic acid end can be realized in the form of an amide bond, and the aldehyde group of intermediate 2 can be conveniently oxidized into carboxylic acid and derivatized. The key to the synthesis of intermediate 2 is the biaryl structure, which can be prepared via intermediates 3 and 4 using Suzuki coupling reactions.

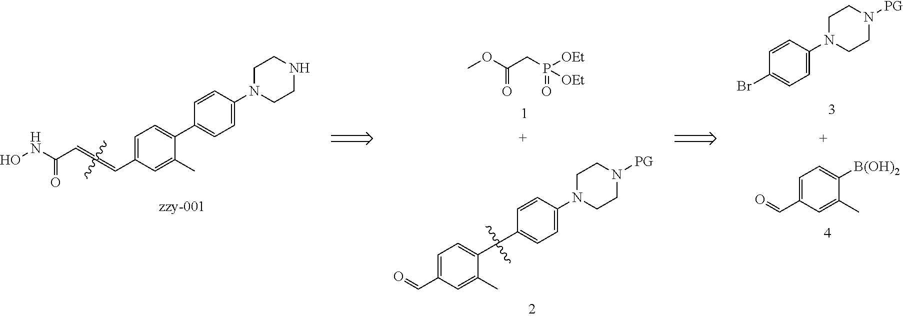

The present invention has been successively tested many times, and now a part of the test results are used as a reference to further describe the present invention in detail, and a detailed description will be given below in conjunction with specific embodiments.

### Embodiment 1

Wherein the synthesis of the intermediate 3 can be completed by the following several routes according to different requirements of the substrate structure:

### Route I: N-alkylation Reaction as a Critical Step

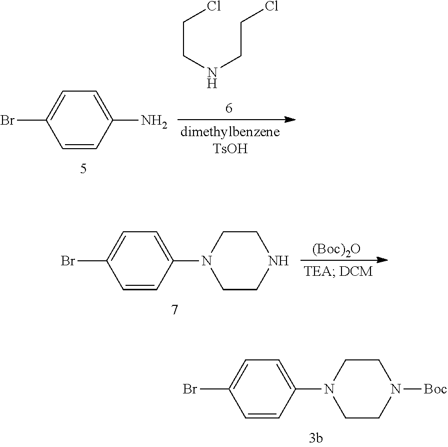 [SMILES: "(Boc)2O" ; ClCCNCCCl ; Nc1ccc(Br)cc1 ; Brc1ccc(N2CCNCC2)cc1 ; CC(C)(C)OC(=O)N1CCN(c2ccc(Br)cc2)CC1]

P-bromoaniline 5 (1.5 eq.), bis (2-chloroethyl) amine hydrochloride 6 (1 eq.) and a catalytic amount of p-toluenesulfonic acid were dissolved in xylene and refluxed. After the end of the TLC tracking reaction, cooled to room temperature and filtered the collected products. The filter cake was washed with water, and then transferred into a round bottom flask, water was added to heat up and reflux to dissolve and clarify, cooled to room temperature again to precipitate solids. Product 7 was obtained by filtering.

Compound 7 (1 eq.) was dissolved in dichloromethane, triethylamine (3 eq.) and Boc anhydride (2 eq.) were added successively and stirred at room temperature overnight. After the reaction was finished, water was added to quench the reaction. Extracted three times with dichloromethane, combined with the organic phases, washed with saturated brine, dried and concentrated. 3b was obtained by column chromatography.

### Route II: Obtaining through Bromo-reduction Route

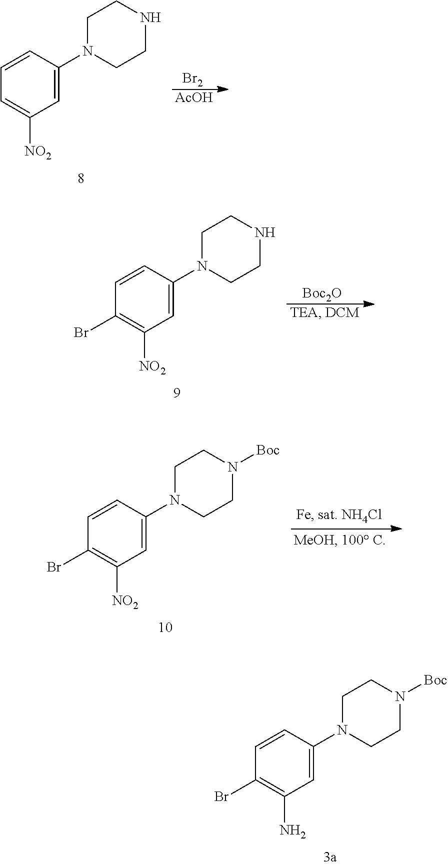

1-(3-nitrophenyl) piperazine 8 (1 eq.) was dissolved in acetic acid and bromine (1.5 eq.) was added dropwise and stirred at 75° C. overnight. After the reaction was finished, the product was collected by filtration, the filter cake was washed with n-hexane, then the filter cake was transferred to a round bottom flask, methanol was added, residual bromine was removed by rotary evaporation, and the product 9 was obtained by vacuum drying. Compound 9 (1 eq.) was dissolved in dichloromethane, triethylamine (3 eq.) and Boc anhydride (2 eq.) were added successively and stirred at room temperature overnight. After the reaction was finished, water was added to quench the reaction, and then extracted with dichloromethane for three times, combined with the organic phases, washed with saturated brine, dried and concentrated. Product 10 was obtained by column chromatography. A round bottom flask was charged with iron powder (3 eq.) and saturated ammonium chloride solution and heated up to 100° C. Compound 10 (1 eq.) was dissolved in methanol and added dropwise into a round bottom flask. Stirring at reflux for 2 hours, cooling to room temperature after the reaction was finished, water was added to quench the reaction, then extracted with dichloromethane for three times, combined with the organic phases, washed with saturated brine, dried and concentrated. 3a was obtained by column chromatography.

### Route III: Aromatic Amination as a Key Step

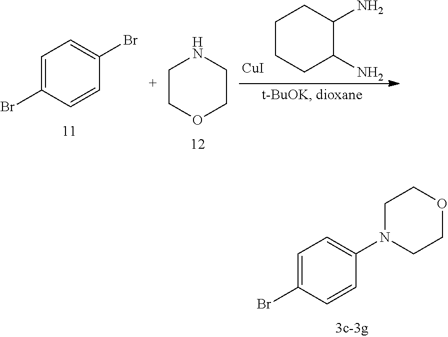

Morpholine 12 (1.1 eq.), p-dibromobenzene 11 (1 eq.) and 2% cuprous iodide were dissolved in 1,4-dioxane and potassium tert-butoxide (2 eq.) and 10% cyclohexanediamine were added and stirred at reflux temperature overnight. The reaction was quenched with saturated ammonium chloride, then extracted with ethyl acetate for three times and combined with organic phases, washed with saturated brine, dried and concentrated. Compound 3c was obtained by column chromatography. 3d-3g can be prepared in the same manner.

The structure and analytical data of intermediate 3 are as follows:

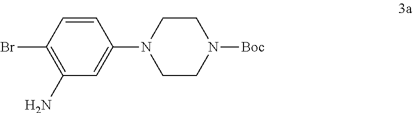 [SMILES: CC(C)(C)OC(=O)N1CCN(c2ccc(Br)c(N)c2)CC1]

¹H NMR (500 MHz, MeOH-d4) δ 7.18-7.16 (d, 1H), 6.46-6.44 (d, 1H), 6.27-6.24 (m, 1H), 3.53 (sbr, 4H), 3.06-3.03 (m, 4H), 1.49 (s, 9H) ppm; ESI-MS obsd 356.0965, calcd 356.0968 [(M+H)⁺, M=C₁₅H₂₂BrN₃O₂].

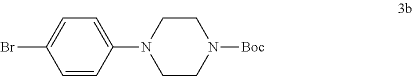 [SMILES: CC(C)(C)OC(=O)N1CCN(c2ccc(Br)cc2)CC1]

¹H NMR (400 MHz, CDCl₃) δ 7.36-7.33 (m, 2H), 6.80-6.77 (m, 2H), 3.58-3.55 (m, 4H), 3.11-3.07 (m, 4H), 1.48 (s, 9H) ppm; ESI-MS obsd 341.0859, calcd 341.0859 [(M+H)⁺, M=C₁₅H₂₁BrN₂O₂].

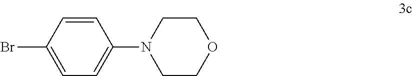 [SMILES: Brc1ccc(N2CCOCC2)cc1]

¹H NMR (300 MHz, CDCl₃) δ 7.48-7.36(m, 2H), 6.71-6.52 (m, 2H), 3.71 (t, 4H), 2.99 (t, 4H) ppm; ESI-MS obsd 242.0177, calcd 242.0175 [(M+H)⁺, M=C₁₀H₁₂BrNO].

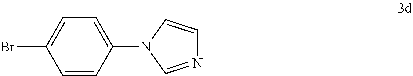 [SMILES: Brc1ccc(-n2ccnc2)cc1]

¹H NMR (300 MHz, CDCl₃) δ 7.26 (s, 1H), 7.27 (s, 1H), 7.36-7.25 (m, 2H), 7.67-7.60 (m, 2H), 7.87 (s, 1H) ppm; ESI-MS obsd 222.9865, calcd 222.9865 [(M+H)⁺, M=C₉H₇BrN₂].

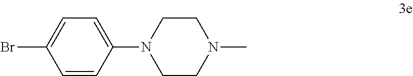

¹H NMR (500 MHz, CDCl₃) δ7.34-7.31 (d, 2H), 6.79-6.73 (d, 2H), 3.26-3.22 (t, 4H), 2.59-2.51 (t, 4H), 2.34 (s, 3H) ppm; ESI-MS obsd 254.0494, calcd 255.0491 [(M+H)⁺, M=C₁₁H₁₅BrN₂].

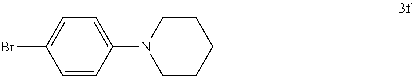 [SMILES: Brc1ccc(N2CCCCC2)cc1]

¹H NMR (400 MHz, CDCl₃) δ 7.31-7.27 (m, 2H), 6.81-6.77 (m, 2H), 3.13-3.08 (m, 4H), 1.74-1.67 (m, 4H), 1.58-1.54 (m, 2H) ppm; ESI-MS obsd 240.0383, calcd 240.0382 [(M+H)⁺, M=C₁₁H₁₄BrN].

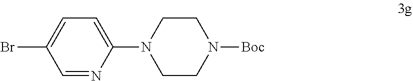 [SMILES: CC(C)(C)OC(=O)N1CCN(c2ccc(Br)cn2)CC1]

¹H NMR (300 MHz, CDCl₃) δ8.20 (s, 1H), 7.56-7.54 (d, 1H), 6.55-6.54 (d, 1H), 3.53-3.50 (m, 8H),1.49 (s, 9H) ppm; ESI-MS obsd 342, calcd 342 [(M+H)⁺, M=C₁₄H₂₀BrN₃O₂].

### Embodiment 2

Intermediate 4 can be obtained by using commercially available borate-based compounds, such as intermediate 4a, or obtained by borylation of properly protected and substituted 4-bromobenzaldehyde under transition metal catalysis, for example, the synthesis route of intermediate 4b was as follows:

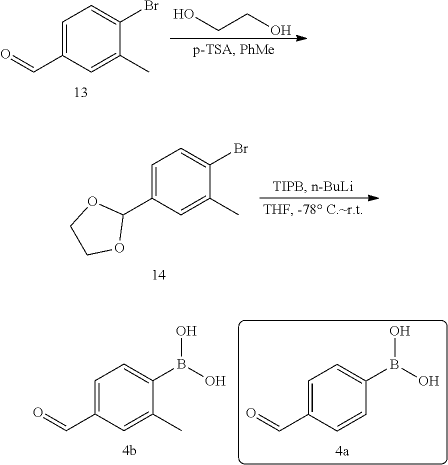

4-bromo-3-tolualdehyde 13 (1 eq.) was dissolved in toluene, ethylene glycol (4 eq.) and catalytic amount of p-toluenesulfonic acid were added successively, connected with Dean-Stark device, and refluxed for 20 hours. After the reaction was finished, cooled to room temperature, poured the reaction mixture into saturated sodium bicarbonate solution, then extracted with ethyl acetate for three times, combined the organic phase, then washed with saturated salt water, dried and concentrated. Product 14 was obtained by column chromatography. Compound 14 (1 eq.) was dissolved in dry tetrahydrofuran, cooled to −78° C. under the protection of nitrogen, n-butyl lithium (1.2 eq.) was added, stirred for half an hour, triisopropyl borate (1.2 eq.) was added to the reaction system, stirred for 1 hour, slowly returned to room temperature and stirred overnight. After the reaction was finished, the reaction was thoroughly quenched by adding 3N hydrochloric acid solution and stirring for 3 hours, then extracted with ethyl acetate for three times, combined with the organic phases, washed with saturated brine, dried and concentrated. 4b was obtained by column chromatography.

Analytical data for intermediate 4b are ¹H NMR (500 MHz, DMSO-d6) δ9.94 (s, 1H), 8.27 (s, 2H), 7.63-7.61 (d, 2H), 7.59-7.58 (d, 1H), 2.44 (s, 3H); ESI-MS obsd 187.0537, calcd 187.0537 [(M+Na)⁺, M=C₈H₉BO₃].

### Synthesis of Intermediate 2

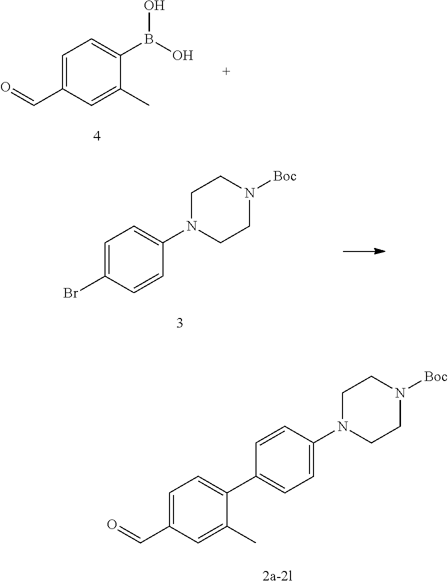 [SMILES: Cc1cc(C=O)ccc1B(O)O ; CC(C)(C)OC(=O)N1CCN(c2ccc(Br)cc2)CC1 ; Cc1cc(C=O)ccc1-c1ccc(N2CCN(C(=O)OC(C)(C)C)CC2)cc1]

Intermediate 3 (1 eq.), intermediate 4 (1.3 eq.) and potassium carbonate (4.5 eq.) were dissolved in a mixed solvent of ethylene glycol dimethyl ether and water (V/V=1:1), and then 5% bis tri-phenyl-phosphonium palladium dichloride catalyst was added and reacted at 80° C. for 6 h under the protection of nitrogen. After completion, cooled to room temperature, the reaction was quenched with water, extracted with ethyl acetate for three times, combined organic phases, washed with saturated brine, dried, and concentrated. Compounds 2a -2l were obtained by column chromatography.

The structure and analytical data for intermediates 2a-2l are as follows:

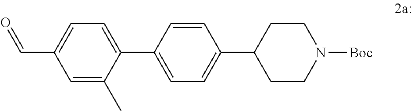 [SMILES: Cc1cc(C=O)ccc1-c1ccc(C2CCN(C(=O)OC(C)(C)C)CC2)cc1]

¹H NMR(400 MHz, MeOH-d4) δ9.96 (s, 1H), 7.79 (s, 1H), 7.75-7.73 (m, 1H), 7.38-7.36 (d, 1H), 7.27-7.24 (m, 2H), 7.07-7.04 (m, 2H), 3.60-3.59 (m, 4H), 3.21-3.18 (m, 4H), 2.35(s, 3H), 1.49 (s, 9H) ppm; ESI-MS obsd 381.2172, calcd 381.2173 [(M+H)⁺, M=C₂₃H₂₈N₂O₃].

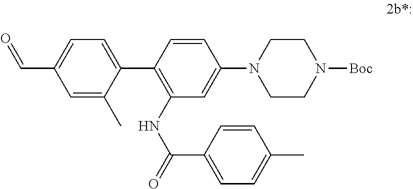 [SMILES: Cc1ccc(C(=O)Nc2cc(N3CCN(C(=O)OC(C)(C)C)CC3)ccc2-c2ccc(C=O)cc2C)cc1]

¹H NMR (400 MHz, CDCl₃) δ10.07 (s, 1H), 8.29-8.28 (d, 1H), 7.86 (s, 1H), 7.83-7.81 (d, 1H), 7.77-7.75 (d, 1H), 7.46-7.43 (m, 2H), 7.36-7.34 (d, 2H), 7.17-7.15 (d, 2H), 7.08-7.05 (d, 1H), 6.78-6.76 (m, 1H), 3.63-3.60 (m, 4H), 3.30-3.28 (m, 4H), 2.35 (s, 3H), 2.23 (s, 3H), 1.50 (s, 9H) ppm; ESI-MS obsd 514.2701, calcd 514.2700 [(M+H)⁺, M=C₃₁H₃₆N₃O₄].

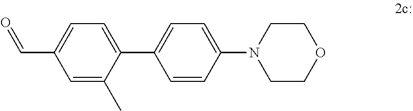 [SMILES: Cc1cc(C=O)ccc1-c1ccc(N2CCOCC2)cc1]

¹H NMR (300 MHz, CDCl₃) δ10.04-10.02 (d, 1H), 7.80-7.73 (m, 2H), 7.43-7.28 (m, 3H), 7.02-6.98 (d, 1H), 6.86-6.83 (d, 1H), 3.93-3.88 (m, 4H), 3.27-3.20 (m, 4H), 2.40-2.37 (d, 3H) ppm; ESI-MS obsd 282.1491, calcd 282.1489 [(M+H)⁺, M=C₁₈H₁₉NO₂].

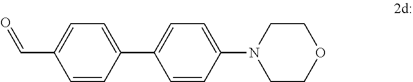 [SMILES: O=Cc1ccc(-c2ccc(N3CCOCC3)cc2)cc1]

¹H NMR (300 MHz, CDCl₃) δ10.08-10.04 (d, 1H), 7.98-7.93 (m, 2H), 7.77-7.73 (m, 2H), 7.63-6.61 (m, 1H), 7.17-7.15 (m, 1H), 7.04-6.98 (m, 2H), 3.93-3.90 (m, 4H), 3.28-3.25 (m, 4H) ppm; ESI-MS obsd 268.1330, calcd 268.1332 [(M+H)⁺, M=C₁₇H₁₇NO₂].

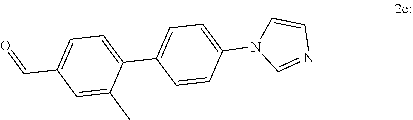 [SMILES: Cc1cc(C=O)ccc1-c1ccc(-n2ccnc2)cc1]

¹H NMR (300 MHz, CDCl₃) δ8.07 (s, 1H), 7.95 (s, 1H), 7.82-7.78 (m, 2H), 7.52-7.39 (m, 7H), 2.39 (s, 3H) ppm; ESI-MS obsd 263.1180, calcd 263.1179 [(M+H)⁺, M=C₁₇H₁₄N₂O].

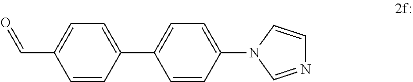 [SMILES: O=Cc1ccc(-c2ccc(-n3ccnc3)cc2)cc1]

¹H NMR (300 MHz, CDCl₃) δ10.07 (s, 1H), 8.00-7.94 (m, 3H), 7.84-7.41 (m, 4H), 7.70-7.63 (m, 1H), 7.53 (s, 1H), 7.50-7.43 (m, 1H), 7.35 (s, 1H) ppm; ESI-MS obsd 249.1026, calcd 249.1022 [(M+H)⁺, M=C₁₆H₁₂N₂O].

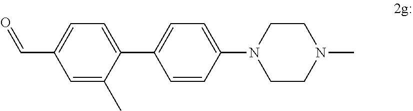 [SMILES: Cc1cc(C=O)ccc1-c1ccc(N2CCN(C)CC2)cc1]

¹H NMR (300 MHz, CDCl₃) δ10.03-10.01 (d, 1H), 7.77-7.72 (m, 2H), 7.42-7.38 (m, 1H), 7.28-7.25 (d, 1H), 7.02-6.95 (m, 2H), 6.86-6.80 (m, 1H), 3.33-3.27 (m, 4H), 2.66-2.61 (m, 4H), 2.40-2.36 (m, 6H) ppm; ESI-MS obsd 295.1806, calcd 295.1805 [(M+H)⁺, M =C₁₉H₂₂N₂O].

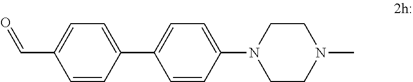 [SMILES: CN1CCN(c2ccc(-c3ccc(C=O)cc3)cc2)CC1]

¹H NMR (300 MHz, CDCl₃) δ10.07-10.03 (d, 1H), 7.97-7.91 (m, 2H), 7.77-7.72 (m, 2H), 7.61-7.58 (m, 1H), 7.41-7.34 (m, 1H), 7.18-7.09 (m, 1H), 7.04-7.01 (m, 1H), 3.35-3.31 (m, 4H), 2.67-2.62 (m, 4H), 2.40 (s, 3H) ppm; ESI-MS obsd 281.1651, calcd 281.1648 [(M+H)⁺, M=C₁₈H₂₀N₂O].

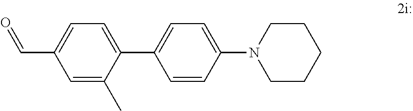 [SMILES: Cc1cc(C=O)ccc1-c1ccc(N2CCCCC2)cc1]

¹H NMR (300 MHz, CDCl₃) δ10.04-10.02 (d, 1H), 7.79-7.73 (m, 2H), 7.44-7.40 (m, 1H), 7.36-7.31 (m, 1H), 7.03-6.97 (m, 2H), 6.88-6.77 (d, 1H), 3.28-3.20 (m, 4H), 2.41-2.38 (d, 3H), 1.80-1.73 (m, 4H), 1.65-1.61 (m, 2H) ppm; ESI-MS obsd 280.1695, calcd 280.1696 [(M+H)⁺, M=C₁₉H₂₁NO].

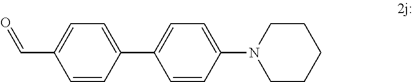 [SMILES: O=Cc1ccc(-c2ccc(N3CCCCC3)cc2)cc1]

¹H NMR (300 MHz, CDCl₃) δ10.07-10.03 (d, 1H), 7.97-7.91 (m, 2H), 7.78-7.73 (m, 2H), 7.61-7.58 (d, 1H), 7.40-7.18 (m, 1H), 7.11-7.01 (m, 2H), 3.30-3.24 (m, 4H), 1.78-1.72 (m, 4H), 1.66-1.63 (m, 2H) ppm; ESI-MS obsd 266.1541, calcd 266.1539 [(M+H)⁺, M=C₁₈H₁₉NO].

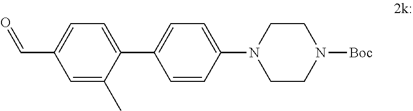 [SMILES: Cc1cc(C=O)ccc1-c1ccc(N2CCN(C(=O)OC(C)(C)C)CC2)cc1]

¹H NMR (300 MHz, CDCl₃) δ10.02 (s, 1H), 8.21 (s, 1H), 7.79-7.74 (m, 2H), 7.54-7.51 (d, 1H), 7.39-7.36 (d, 1H), 6.75-6.72 (d, 1H), 3.61 (s, 8H), 2.40 (s, 3H), 1.51 (s, 9H) ppm; ESI-MS obsd 382.2129, calcd 382.2125 [(M+H)⁺, M=C₂₂H₂₇N₃O₃].

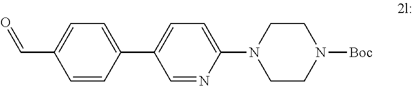 [SMILES: CC(C)(C)OC(=O)N1CCN(c2ccc(-c3ccc(C=O)cc3)cn2)CC1]

¹H NMR (300 MHz, CDCl₃) δ10.01 (s, 1H), 8.51 (s, 1H), 7.93-7.91 (d, 2H), 7.79-7.76 (d, 1H), 7.68-7.66 (d, 2H), 6.73-6.71 (d, 1H), 3.63-3.56 (m, 8H), 1.49 (s, 9H) ppm; ESI-MS obsd 368.1970, calcd 368.1969 [(M+H)⁺, M=C₂₁H₂₅N₃O₃].

Wherein the preparation of intermediate 2b still requires a one-step amidation reaction after Suzuki coupling, as follows:

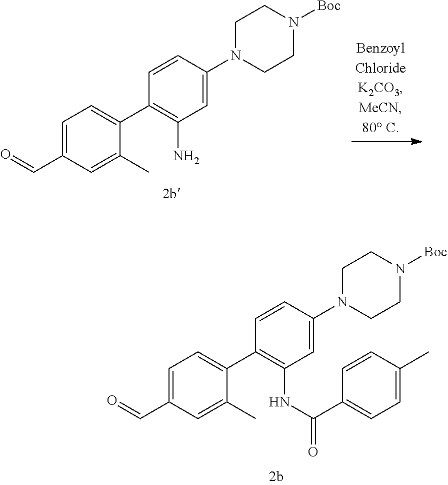 [SMILES: Cc1cc(C=O)ccc1-c1ccc(N2CCN(C(=O)OC(C)(C)C)CC2)cc1N ; Cc1ccc(C(=O)Nc2cc(N3CCN(C(=O)OC(C)(C)C)CC3)ccc2-c2ccc(C=O)cc2C)cc1]

The product 2b′ (1 eq.) of the Suzuki reaction was dissolved in acetonitrile, potassium carbonate (2 eq.) was added, cooled to 0° C. and p-methylbenzoyl chloride (1.1 eq.) was slowly added dropwise, then heated up to 80° C. and stirred for half an hour to finish the reaction. Cooled to room temperature, and the reaction was quenched with water, then extracted with ethyl acetate for three times, combined organic phases, washed with saturated brine, dried, and concentrated. Compound 2b was obtained by column chromatography.

### Embodiment 3

Trimethyl phosphonoacetate (1.1 eq.) was dissolved in dry tetrahydrofuran, cooled to −20° C., potassium hexamethyldisilazide (1.1 eq.) in tetrahydrofuran (1.0 M) was added dropwise, stirred for 20-30 min, then intermediate 2 (1 eq.) in tetrahydrofuran was added dropwise, stirred for 1 h and the reaction was finished. The reaction was quenched with water, then extracted with ethyl acetate for three times and combined with organic phases, washed with saturated brine, dried and concentrated. Compound 15a was obtained by column chromatography. In this way it was also possible to prepare 15b -15c and 15g -15p, according to the following formula:

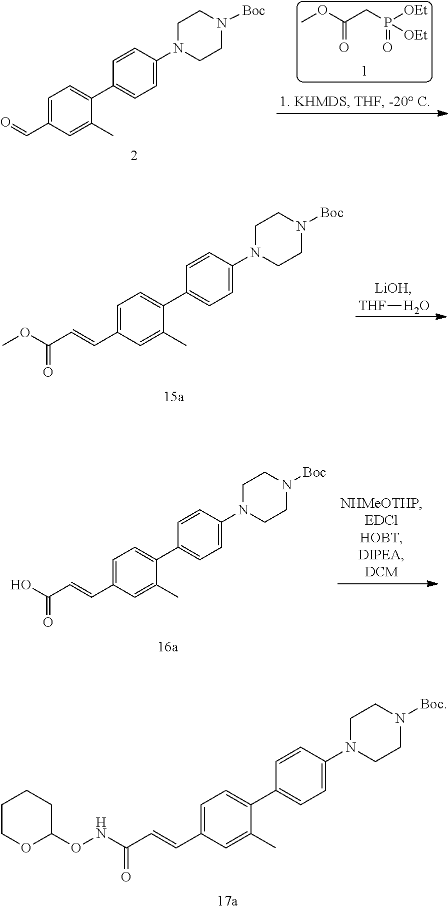

The structure and analytical data of intermediate 15 are as follows:

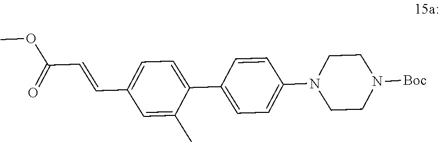 [SMILES: COC(=O)/C=C/c1ccc(-c2ccc(N3CCN(C(=O)OC(C)(C)C)CC3)cc2)c(C)c1]

¹H NMR(500 MHz, CDCl₃) δ 7.71-7.68 (d, 1H), 7.41-7.40 (m, 2H), 7.25-7.23 (d, 3H), 7.00-6.96 (d, 2H), 6.46-6.43 (d, 1H), 3.81 (s, 3H), 3.61-3.59 (m, 4H), 3.21-3.20 (m, 4H), 2.31 (s, 3H), 1.49 (s, 9H) ppm; ESI-MS obsd 437.2442, calcd 437.2435 [(M+H)⁺, M=C₂₆H₃₂N₂O₄].

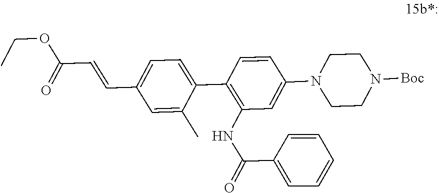 [SMILES: CCOC(=O)/C=C/c1ccc(-c2ccc(N3CCN(C(=O)OC(C)(C)C)CC3)cc2NC(=O)c2ccccc2)c(C)c1]

¹H NMR (500 MHz, CDCl₃) δ8.32 (sbr, 1H), 7.73-7.70 (d, 1H), 7.59 (s, 1H), 7.51 (s, 1H), 7.49-7.45 (m, 4H), 7.37-7.34 (m, 2H), 7.29-7.27 (d, 1H), 7.08-7.06 (d, 1H), 6.77-6.75 (m, 1H), 6.51-6.48 (d, 1H), 4.31-4.27 (m, 2H), 3.60 (s, 4H), 3.28 (s, 4H), 2.17 (s, 3H), 1.50 (s, 9H), 1.37-1.34 (m, 3H) ppm; ESI-MS obsd 570.2964, calcd 570.2962 [(M+H)⁺, M=C₃₄H₃₉N₃O₅].

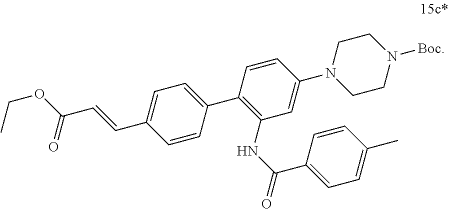 [SMILES: CCOC(=O)/C=C/c1ccc(-c2ccc(N3CCN(C(=O)OC(C)(C)C)CC3)cc2NC(=O)c2ccc(C)cc2)cc1]

¹H NMR (300 MHz, CDCl₃) δ8.34 (sbr, 1H), 7.77-7.71 (d, 1H), 7.59 (s, 1H), 7.53-7.49 (d, 2H), 7.39-7.37 (d, 2H), 7.31 (s, 1H), 7.19-7.16 (d, 2H), 7.10-7.07 (d, 1H), 6.78-6.75 (d, 1H), 6.55-6.49 (m, 1H), 4.35-4.28 (m, 2H), 3.65-3.61 (m, 4H), 3.31-3.28 (m, 4H), 2.37 (s, 3H), 2.18 (s, 3H), 1.52 (s, 9H), 1.41-1.36 (m, 3H) ppm; ESI-MS obsd 584.3121, calcd 584.3119 [(M+H)⁺, M=C₃₅H₄₁N₃O₅].

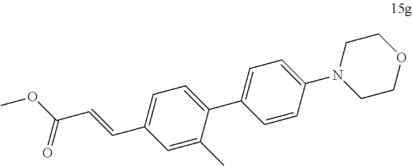 [SMILES: COC(=O)/C=C/c1ccc(-c2ccc(N3CCOCC3)cc2)c(C)c1]

¹H NMR (300 MHz, CDCl₃) δ7.76-7.70 (m, 1H), 7.44-7.40 (d, 2H), 7.35-7.27 (m, 2H), 7.00-6.83 (m, 2H), 6.86-6.83 (d, 1H), 6.52-6.45 (m, 1H), 3.93-3.88 (m, 4H), 3.84-3.83 (m, 3H), 3.26-3.20 (m, 4H), 2.34-2.30 (m, 3H) ppm; ESI-MS obsd 338.1753, calcd 338.1751 [(M+H)⁺, M=C₂₁H₂₃NO₃].

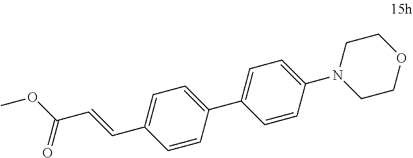 [SMILES: COC(=O)/C=C/c1ccc(-c2ccc(N3CCOCC3)cc2)cc1]

¹H NMR (300 MHz, CDCl₃) δ7.78 (s, 1H), 7.72 (s, 1H), 7.62-7.61 (d, 4H), 7.57 (s, 1H),7.02-7.00 (d, 2H), 6.51-6.46 (d, 1H), 3.93-3.90 (m, 4H), 3.84 (s, 3H), 3.26-3.23 (m, 4H); ESI-MS obsd 324.1596, calcd 324.1594 [(M+H)⁺, M=C₂₀H₂₁NO₃].

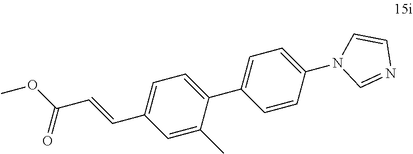 [SMILES: COC(=O)/C=C/c1ccc(-c2ccc(-n3ccnc3)cc2)c(C)c1]

¹H NMR (300 MHz, CDCl₃) δ7.91 (s, 1H), 7.72-7.67 (d, 1H), 7.44-7.41 (m, 6H), 7.33 (s, 1H), 7.26-7.22 (m, 2H), 6.50-6.44 (d, 1H), 3.74 (s, 3H), 2.39 (s, 3H) ppm; ESI-MS obsd 319.1440, calcd 319.1441 [(M+H)⁺, M=C₂₀H₁₈N₂O₂].

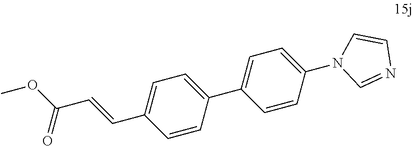 [SMILES: COC(=O)/C=C/c1ccc(-c2ccc(-n3ccnc3)cc2)cc1]

¹H NMR (300 MHz, CDCl₃) δ7.94 (s, 1H), 7.79-7.75 (d, 1H), 7.74-7.73 (d, 1H), 7.66 (s, 4H), 7.50-7.49 (m, 4H), 7.36 (s, 1H), 6.55-6.49 (d, 1H), 3.86-3.85 (s, 3H); ESI-MS obsd 305.1287, calcd 305.1285 [(M+H)⁺, M=C₁₉H₁₆N₂O₂].

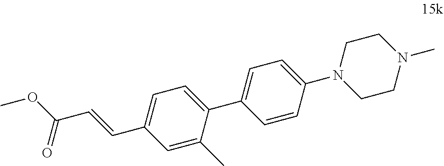

¹H NMR (300 MHz, CDCl₃) δ7.75-7.69 (d, 1H), 7.43-7.40 (d, 2H), 7.28-7.24 (m, 2H), 7.01-7.00 (d, 2H), 6.87-6.81 (m, 1H), 6.51-6.44 (m, 1H), 3.83 (s, 3H), 3.40-3.26 (m, 4H), 2.64-2.63 (m, 4H), 2.40-2.39 (m, 3H), 2.34-2.31 (m, 3H) ppm; ESI-MS obsd 351.2065, calcd 351.2067 [(M+H)⁺, M=C₂₂H₂₆N₂O₂].

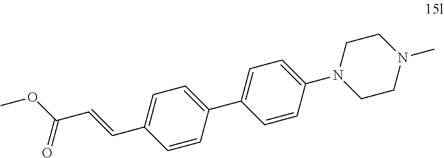

¹H NMR (300 MHz, CDCl₃) δ7.78-7.71 (m, 1H), 7.61-7.57 (m, 4H), 7.39-7.34 (d, 1H), 7.15-7.10 (m, 1H), 7.05-6.96 (m, 2H), 6.52-6.44 (m, 1H), 3.84-3.83 (m, 3H), 3.40 (s, 4H), 2.66-2.63 (m, 4H), 2.40 (s, 3H) ppm; ESI-MS obsd 337.1911, calcd 337.1911 [(M+H)⁺, M=C₂₁H₂₄N₂O₂].

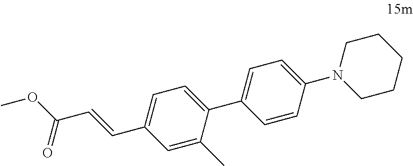 [SMILES: COC(=O)/C=C/c1ccc(-c2ccc(N3CCCCC3)cc2)c(C)c1]

¹H NMR (300 MHz, CDCl₃) δ7.77-7.71 (m, 1H), 7.43-7.41 (d, 2H), 7.31-7.27 (m, 2H), 7.02-6.95 (m, 2H), 6.89-6.77 (m, 1H), 6.52-6.45 (m, 1H), 3.84 (s, 3H), 3.27-3.20 (m, 4H), 2.54-2.52 (s, 3H), 1.80-1.73 (m, 4H), 1.67-1.61 (m, 2H) ppm; ESI-MS obsd 336.1960, calcd 336.1958 [(M+H)⁺, M=C₂₂H₂₅NO₂].

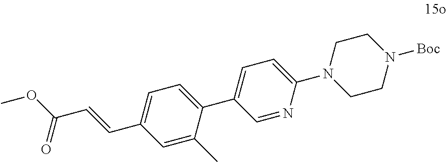 [SMILES: COC(=O)/C=C/c1ccc(-c2ccc(N3CCN(C(=O)OC(C)(C)C)CC3)nc2)c(C)c1]

¹H NMR (300 MHz, CDCl₃) δ8.21-8.20 (d, 1H), 7.74-7.69 (d, 1H), 7.53-7.41 (m, 3H), 7.25-7.23 (d, 1H), 6.74-6.71 (d, 1H), 6.50-6.45 (d, 1H), 3.83 (s, 3H), 3.60 (s, 8H), 2.34 (s, 3H), 1.52 (s, 9H) ppm; ESI-MS obsd 438.2387, calcd 438.2387 [(M+H)⁺, M=C₂₅H₃₁N₃O₄].

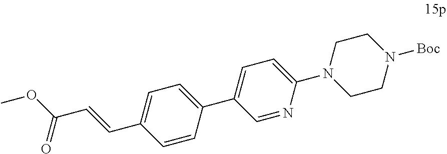 [SMILES: COC(=O)/C=C/c1ccc(-c2ccc(N3CCN(C(=O)OC(C)(C)C)CC3)nc2)cc1]

¹H NMR (300 MHz, CDCl₃) δ8.52-8.51 (d, 1H), 7.75-7.70 (m, 2H), 7.61-7.55 (m, 3H), 7.30-7.28 (m, 1H), 6.75-6.72 (d, 1H), 6.52-6.46 (d, 1H), 3.85 (s, 3H), 3.62 (s, 8H), 1.51 (s, 9H) ppm; ESI-MS obsd 424.2229, calcd 424.2231 [(M+H)⁺, M=C₂₄H₂₉N₃O₄].

### Embodiment 4

Intermediate 15 (1 eq.) was dissolved in a mixed solvent of tetrahydrofuran and water (V/V=1:1), lithium hydroxide (5 eq.) was added, stirred at room temperature for 2-3 hours, and the reaction was finished. The tetrahydrofuran was removed by rotary evaporation, the pH was adjusted to 2-3 with 1 N hydrochloric acid solution, then extracted with ethyl acetate for three times and combined with organic phases, washed with saturated brine, dried and concentrated. Compound 16 was obtained by column chromatography. 16 (1 eq.), O-(tetrahydro-2H-pyran-2-yl) hydroxylamine (2 eq.), EDCI (2 eq.), HOBt (2 eq.) was added into a round bottom flask, dissolved by addition of dichloromethane, and DIPEA (4 eq.) was added and stirred at room temperature for 1.5 hours. After the reaction was finished, water was used to quench the reaction, and then extracted with dichloromethane for three times, combined with the organic phases, washed with saturated brine, dried and concentrated. Compound 17a was obtained by column chromatography. In this way it was also possible to prepare 17b -17c and 17g -17p.

Intermediate 2c (1 eq.) and 2-methyl-2-butene (20 eq.) were dissolved in tert-butanol, and a mixed solution of sodium chlorite (10 eq.) and 20% sodium dihydrogen phosphate was added to the reaction system, stirred at room temperature overnight, and the reaction was finished. The reaction was quenched by the addition of saturated sodium sulfite, stirred for 15 min and monitored for pH to 7, then extracted with ethyl acetate for three times and combined with organic phases, washed with saturated brine, dried and concentrated. Compound 18 was obtained by column chromatography. Intermediate 18 (1 eq.), o-phenylenediamine 19 (1.2 eq.) and HOBt (2 eq.) were dissolved in dichloromethane and DIPEA (4 eq.) was added dropwise followed by EDCI (2 eq.) and stirred at room temperature overnight. After the reaction was finished, saturated ammonium chloride solution was used to quench the reaction, and then extracted with dichloromethane for three times, combined with the organic phases, washed with saturated brine, dried and concentrated. Compounds 17d-17f were obtained by column chromatography, and the reaction formula was as follows:

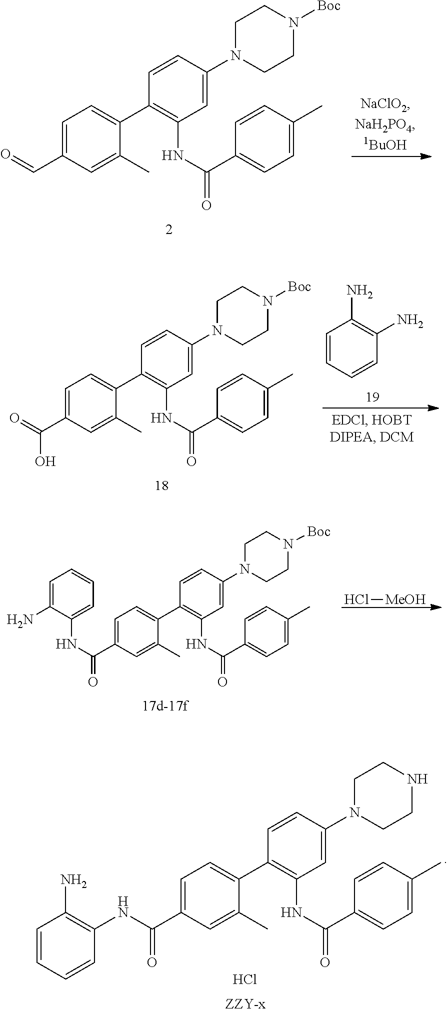

The structure and analytical data of 17a -17p are as follows:

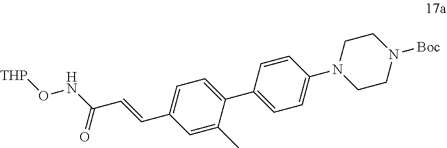

¹H NMR (500 MHz, CDCl₃) δ8.32 (sbr, 1H), 7.76-7.72 (d, 1H), 7.40-7.37 (d, 2H), 7.25-7.23 (m, 3H), 6.98-6.96 (d, 2H), 5.01 (s, 1H), 4.00-3.96 (m, 1H), 3.68-3.66 (d, 1H), 3.61-3.59 (m, 4H), 3.21-3.19 (m, 4H), 2.31 (s, 3H), 1.87-1.62 (m, 6H), 1.49 (s, 9H) ppm; ESI-MS obsd 522.2966, calcd 522.2962 [(M+H)⁺, M=C₃₀H₃₉N₃O₅].

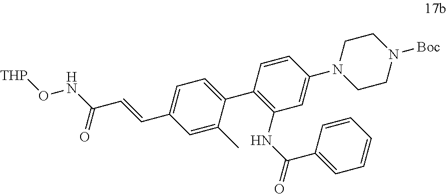

¹H NMR (400 MHz, CDCl₃) δ8.34-8.33 (d, br, 1H), 7.83-7.71 (m, 2H), 7.64-7.62 (m, 1H), 7.57-7.47 (m, 3H), 7.41-7.36 (m, 1H), 7.28-7.25 (m, 5H), 7.11-7.08 (d, 1H), 6.80-6.77 (m, 1H), 5.10-5.04 (m, 1H), 4.70-3.91 (m, 6H), 3.70-3.59 (m, 2H), 3.35-3.25 (m, 2H), 2.18-2.07 (s, 3H), 1.80-1.62 (m, 2H), 1.55-1.52 (s, 9H), 1.50-1.32 (m, 4H) ppm; ESI-MS obsd 641.3334, calcd 641.3334 [(M+H)⁺, M=C₃₇H₄₄N₄O₆].

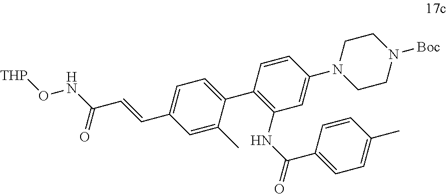

¹H NMR (400 MHz, CDCl₃) δ8.32-8.31 (d, br, 1H), 7.80-7.76 (d, 1H), 7.56 (s, 1H), 7.49-7.46 (m, 2H), 7.36-7.34 (m, 2H), 7.28 (s, 1H), 7.17-7.15 (m, 2H), 7.07-7.05 (d, 1H), 6.76-6.74 (m, 1H), 5.04 (s, 1H), 3.94-3.88 (m, 1H), 3.70-3.67 (m, 1H), 3.62-3.60 (m, 4H), 3.29-3.26 (m, 4H), 2.35 (s, 3H), 2.15 (s, 3H), 1.94-1.72 (m, 6H), 1.50 (s, 9H) ppm; ESI-MS obsd 655.3497, calcd 655.3490 [(M+H)⁺, M=C₃₈H₄₆N₄O₆].

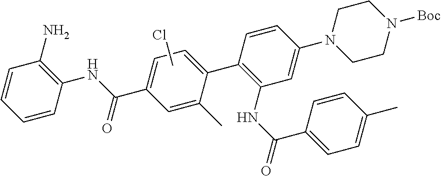

¹H NMR (500 MHz, CDCl₃) δ8.29 (sbr, 1H), 7.92 (s, 2H), 7.83-7.82 (d, 1H), 7.52 (s, 1H), 7.40-7.38 (m, 4H), 7.19-7.17 (d, 2H), 7.14-7.11 (m, 1H), 7.07-7.05 (d, 1H), 6.89-6.88 (m, 2H), 6.78-6.76 (m, 1H), 5.30 (sbr, 1H), 3.62-3.60 (m, 4H), 3.29-3.27 (m, 4H), 2.36 (s, 3H), 2.23 (s, 3H), 1.50 (s, 9H) ppm; ESI-MS obsd 654.2849, calcd 654.2842 [(M+H)⁺, M=C₃₇H₄₀ClN₅O₄].

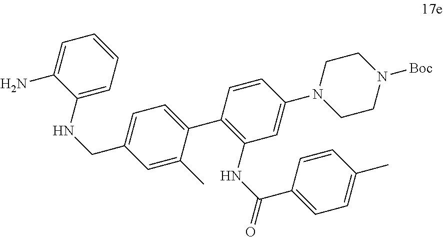 [SMILES: Cc1ccc(C(=O)Nc2cc(N3CCN(C(=O)OC(C)(C)C)CC3)ccc2-c2ccc(CNc3ccccc3N)cc2C)cc1]

¹H NMR (400 MHz, MeOH-d₄) δ7.89-7.88 (d, 1H), 7.81-7.77 (m, 1H), 7.53-7.50 (d, 2H), 7.35-7.33 (m, 1H), 7.23-7.18 (m, 3H), 7.10-7.01 (m, 2H), 6.91-6.89 (m, 1H), 6.79-6.75 (m, 1H), 6.72-6.68 (m, 2H), 6.61-6.58 (m, 2H), 3.72-3.57 (m, 4H), 3.29-3.17 (m, 4H), 2.35 (s, 3H), 2.25 (s, 3H), 1.49 (s, 9H) ppm; ESI-MS obsd 620.3235, calcd 620.3231 [(M+H)⁺, M=C₃₇H₄₁N₅O₄].

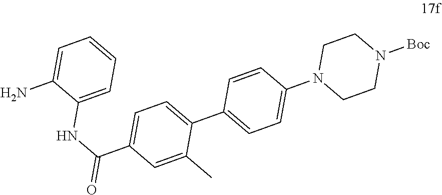 [SMILES: Cc1cc(C(=O)Nc2ccccc2N)ccc1-c1ccc(N2CCN(C(=O)OC(C)(C)C)CC2)cc1]

¹H NMR (500 MHz, CDCl₃) δ7.86 (sbr, 1H), 7.83-7.81 (m, 1H), 7.75-7.72 (m, 1H), 7.37-7.32 (m, 2H), 7.28-7.24 (m, 2H), 7.13-7.08 (m, 1H), 7.01-6.96 (m, 2H), 6.89-6.84 (m, 2H), 3.92 (sbr, 2H), 3.64-3.58 (m, 4H), 3.25-3.17 (m, 4H), 2.37 (s, 3H), 1.50 (s, 9H) ppm; ESI-MS obsd 487.2707, calcd 487.2704 [(M+H)⁺, M=C₂₉H₃₄N₄O₃].

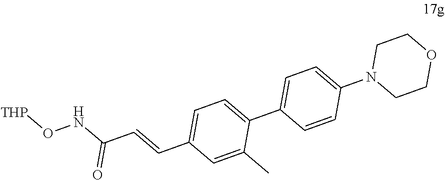

¹H NMR (300 MHz, CDCl₃) δ9.04 (sbr, 1H), 7.79-7.73 (d, 1H), 7.41-7.28 (m, 3H), 7.23 (s, 1H), 6.98-6.90 (m, 2H), 6.84-6.81 (d, 1H), 6.49 (m, 1H), 3.92-3.86 (m, 4H), 3.70-3.60 (m, 2H), 3.23-3.20 (m, 4H), 2.31-2.28 (d, 3H), 1.88-1.56 (m, 6H) ppm; ESI-MS obsd 445.2103, calcd 445.2098 [(M+Na)⁺, M=C₂₅H₃₀N₂O₄].

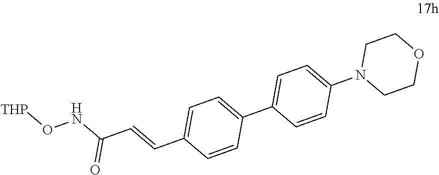

¹H NMR (300 MHz, CDCl₃) δ8.77 (sbr, 1H), 7.83-7.76 (m, 3H), 7.44-7.32 (m, 2H), 7.17-7.10 (m, 2H), 7.05-6.90 (m, 2H), 6.48 (sbr, 1H), 5.05 (br, 1H), 4.50-3.95 (m, 6H),3.90-3.60 (m, 4H), 1.88-1.56 (m, 6H) ppm; ESI-MS obsd 409.2127, calcd 409.2122 [(M+H)⁺, M=C₂₄H₂₈N₂O₄].

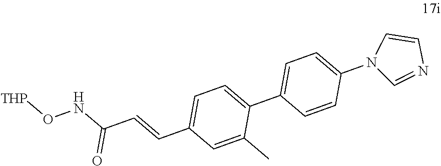

¹H NMR (300 MHz, CDCl₃) δ8.20 (s, 1H), 7.67-7.62 (d, 1H), 7.47-7.40 (m, 8H), 7.28-7.26 (d, 2H), 7.17-7.15 (d, 1H), 4.04 (s, 1H), 3.88-3.85 (m, 2H), 2.24 (s, 3H), 1.70-1.62 (m, 6H) ppm; ESI-MS obsd 404.1889, calcd 404.1969 [(M+H)⁺, M=C₂₄H₂₅N₃O₃].

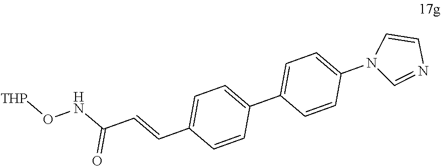

¹H NMR (300 MHz, CDCl₃) δ7.82-7.74 (m, 2H), 7.70-7.62 (m, 4H), 7.60-7.55 (m, 3H), 7.36-7.26 (m, 3H), 6.78-6.73 (m, 1H), 5.04-4.95 (m, 1H), 4.10-3.78 (m, 2H), 1.71-1.60 (m, 6H) ppm; ESI-MS obsd 390.1815, calcd 390.1812 [(M+H)⁺, M=C₂₃H₂₃N₃O₃].

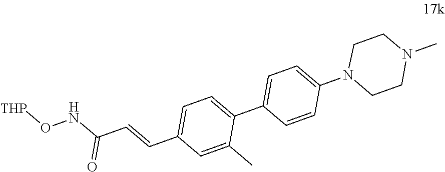

¹H NMR (300 MHz, CDCl₃) δ7.87-7.84 (d, 1H), 7.75-7.63 (d, 2H), 7.35-7.32 (d, 1H), 7.15-7.13 (d, 2H), 6.87-6.78 (m, 2H), 6.56 (s, 1H), 5.08 (s, 1H), 4.00 (s, 1H), 3.62 (s, 1H), 3.37 (m, 4H), 3.05-3.01 (m, 4H), 2.66-2.62 (m, 3H), 2.22 (s, 3H), 1.85-1.59 (m, 6H) ppm; ESI-MS obsd 436.2599, calcd 436.2595 [(M+H)⁺, M=C₂₆H₃₃N₃O₃].

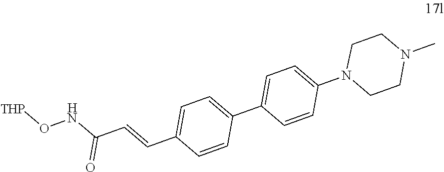

¹H NMR (300 MHz, CDCl₃) δ7.86-7.83 (d, 1H), 7.76-7.71 (d, 1H), 7.65-7.63 (d, 1H), 7.50-7.46 (d, 4H), 7.10-7.02 (m, 1H), 6.87-6.84 (d, 1H), 6.59 (s, 1H), 5.09 (s, 1H), 4.00 (s, 1H), 3.61 (s, 1H), 3.40 (s, 4H), 3.14 (s, 4H), 2.72-2.70 (s, 3H), 1.85-1.60 (m, 6H) ppm; ESI-MS obsd 422.2438, calcd 422.2438 [(M+H)⁺, M=C₂₅H₃₁N₃O₃].

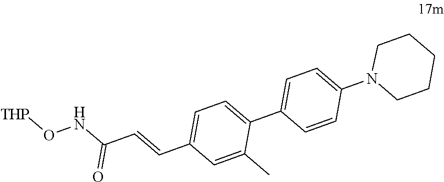

¹H NMR (300 MHz, CDCl₃) δ8.61 (sbr, 1H), 7.79-7.74 (d, 1H), 7.41-7.39 (d, 1H), 7.33-7.30 (d, 1H), 7.26-7.21 (m, 2H), 7.01-6.93 (m, 2H), 6.87-6.76 (m, 1H), 5.04 (s, 1H), 4.04-3.93 (m, 1H), 3.71-3.63 (m, 1H), 3.26-3.21 (m, 4H), 2.33-2.31 (s, 3H), 1.89-1.85 (m, 2H), 1.79-1.70 (m, 6H), 1.63-1.56 (m, 4H) ppm; ESI-MS obsd 421.2424, calcd 421.2486 [(M+H)⁺, M=C₂₆H₃₂N₂O₃].

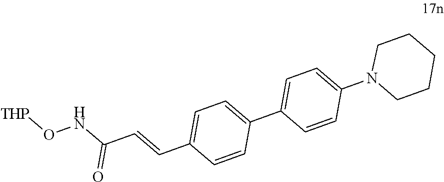

¹H NMR (300 MHz, CDCl₃) δ8.82 (sbr, 1H), 7.80-7.73 (m, 1H), 7.60-7.56 (d, 4H), 7.54-7.51 (d, 1H), 7.36-7.33 (m, 1H), 7.07-6.95 (m, 2H), 6.54-6.46 (m, 1H), 5.05 (s, 1H), 4.01-3.92 (m, 1H), 3.71-3.61 (m, 1H), 3.23 (s, 4H), 1.89-1.85 (m, 2H), 1.75-1.74 (m, 6H), 1.63-1.61 (m, 4H) ppm; ESI-MS obsd 407.2325, calcd 407.2329 [(M+H)⁺, M=C₂₅H₃₀N₂O₃].

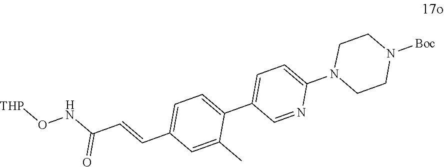

¹H NMR (300 MHz, CDCl₃) δ9.30 (sbr, 1H), 8.17 (d, 1H), 7.77-7.71 (d, 1H), 7.50-7.47 (m, 1H), 7.40 (s, 2H), 7.20-7.17 (d, 1H), 6.72-6.69 (d, 1H), 6.49 (s, 1H), 5.05 (s, 1H), 4.04-3.98 (m, 1H), 3.68-3.65 (d, 1H), 3.53 (s, 8H), 2.34 (s, 3H), 1.87-1.62 (m, 6H), 1.50 (s, 9H) ppm; ESI-MS obsd 545.2720, calcd 545.2734 [(M+Na)⁺, M=C₂₉H₃₈N₄O₅].

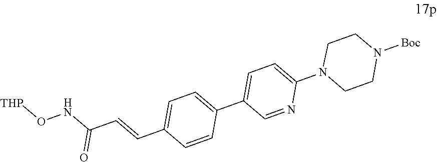

¹H NMR (300 MHz, CDCl₃) δ8.49 (d, 2H), 7.81-7.78 (d, 1H), 7.75 (s, 1H), 7.61-7.54 (m, 4H), 6.75-6.72 (d, 1H), 5.04 (s, 1H), 4.04-3.98 (m, 1H), 3.71-3.67 (d, 1H), 3.60 (s, 8H), 1.89-1.66 (m, 6H), 1.51 (s, 9H) ppm; ESI-MS obsd 531.2564, calcd 531.2578 [(M+Na)⁺, M=C₂₈H₃₆N₄O₅].

### Embodiment 5

Intermediate 17 (e.g. 17a-17p) was dissolved in methanol and then added dropwise to a 2 N hydrogen chloride in methanol (2 eq.), stirred at room temperature for 30 min, most of the methanol was removed by rotary evaporation after the reaction was monitored by TLC, the system was kept clear, ether was added to precipitate a solid, standing was carried out, the solvent was sucked out, and dried to obtain the product ZZY-001 to ZZY-016 series molecules. The above operation can also be done with trifluoroacetic acid and dichloromethane system, and the final product molecules obtained have different carboxylic acid pairing ions.

For example, the reaction formula for the generation of ZZY-001 was as follows:

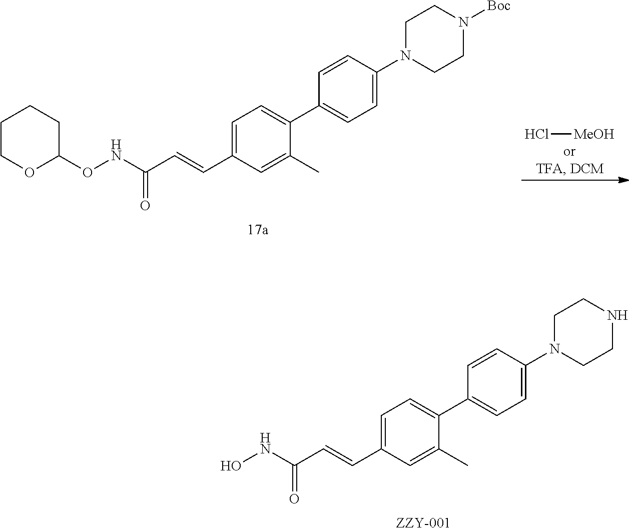

The structure and analytical data of ZZY-001 to ZZY-016 are as follows:

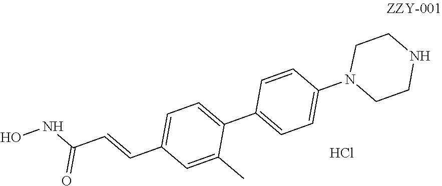 [SMILES: Cc1cc(/C=C/C(=O)NO)ccc1-c1ccc(N2CCNCC2)cc1 ; Cl]

¹H NMR (500 MHz, MeOH-d4) δ7.90 (sbr, 1H), 7.61-7.58 (d, 1H), 7.46-7.42 (m, 2H), 7.29-7.27 (d, 2H), 7.22-7.20 (d, 1H), 7.13-7.12 (m, 2H), 6.50-6.47 (d, 1H), 3.50-3.49 (m, 4H), 3.43-3.42 (m, 4H), 2.28 (s, 3H) ppm; ESI-MS obsd 338.1862, calcd 338.1863 [(M+H)⁺, M=C₂₀H₂₃N₃O₂].

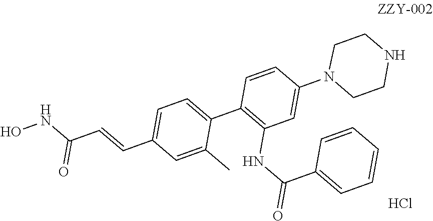 [SMILES: Cc1cc(/C=C/C(=O)NO)ccc1-c1ccc(N2CCNCC2)cc1NC(=O)c1ccccc1 ; Cl]

¹H NMR (500 MHz, MeOH-d4) δ7.58-7.54 (m, 3H), 7.52-7.48 (m, 3H), 7.44-7.37 (m, 3H), 7.24-7.20 (m, 2H), 7.06-7.03 (m, 1H), 6.49-6.45 (d, 1H), 3.54-3.51 (m, 4H), 3.43-3.40 (m, 4H), 2.17 (s, 3H) ppm; ESI-MS obsd 457.2235, calcd 457.2234 [(M+H)⁺, M=C₂₇H₂₈N₄O₃].

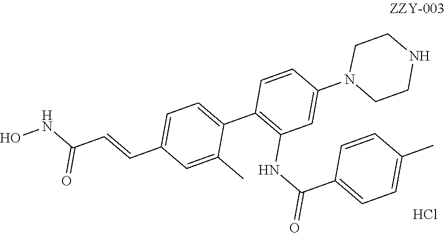 [SMILES: Cc1ccc(C(=O)Nc2cc(N3CCNCC3)ccc2-c2ccc(/C=C/C(=O)NO)cc2C)cc1 ; Cl]

¹H NMR (500 MHz, MeOH-d4) δ7.74 (s, 1H), 7.64-7.60 (d, 1H), 7.48-7.42 (m, 4H), 7.32-7.26 (m, 2H), 7.23-7.15 (m, 3H), 6.57-6.54 (m, 1H), 3.77 (s, 4H), 3.60 (s, 4H), 2.31 (s, 3H), 2.14 (s, 3H) ppm; ESI-MS obsd 471.2390, calcd 471.2391 [(M+H)⁺, M=C₂₈H₃₀N₄O₃].

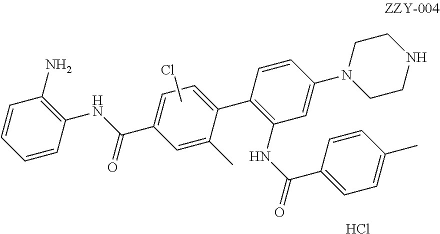

¹H NMR (400 MHz, MeOH-d4) δ 7.61-7.18 (m, 15H), 3.48-3.37 (m, 8H), 2.36 (s, 3H), 2.28 (s, 3H) ppm; ESI-MS obsd 554.2235, calcd 554.2317 [(M+H)⁺, M=C₃₂H₃₂ClN₅O₂].

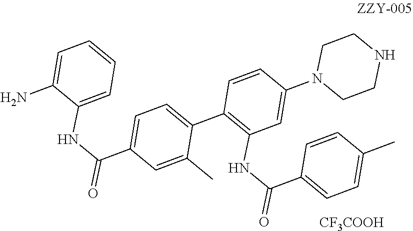 [SMILES: Cc1ccc(C(=O)Nc2cc(N3CCNCC3)ccc2-c2ccc(C(=O)Nc3ccccc3N)cc2C)cc1 ; "CF3COOH"]

¹H NMR (500 MHz, MeOH-d4) δ 7.94 (sbr, 1H), 7.86-7.85 (d, 1H), 7.53-7.51 (d, 2H), 7.38-7.33 (m, 4H), 7.30-7.27 (m, 2H), 7.23-7.20 (m, 2H), 7.09-7.07 (m, 1H), 5.49 (sbr, 1H), 3.54-3.52 (m, 4H), 3.43-3.40 (m, 4H), 2.35 (s, 3H), 2.26 (s, 3H) ppm; ESI-MS obsd 520.2708, calcd 520.2707 [(M+H)⁺, M=C₃₂H₃₃N₅O₂].

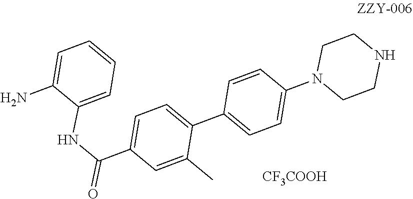 [SMILES: Cc1cc(C(=O)Nc2ccccc2N)ccc1-c1ccc(N2CCNCC2)cc1 ; "CF3COOH"]

¹H NMR (400 MHz, MeOH-d4) δ 7.95 (sbr, 1H), 7.89-7.86 (d, 1H), 7.38-7.29 (m, 5H), 7.26-7.20 (m, 2H), 7.14-7.12 (m, 2H), 3.49-3.48 (m, 4H), 3.42-3.39 (m, 4H), 2.36 (s, 3H) ppm; ESI-MS obsd 387.2183, calcd 387.2179 [(M+H)⁺, M=C₂₄H₂₆N₄O].

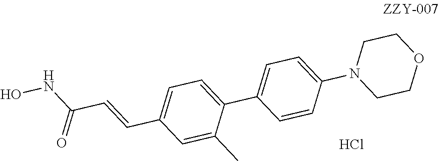 [SMILES: Cc1cc(/C=C/C(=O)NO)ccc1-c1ccc(N2CCOCC2)cc1 ; Cl]

¹H NMR (300 MHz, MeOH-d4) δ7.91-7.82 (m, 2H), 7.61-7.48 (m, 5H), 7.32-7.24 (m, 1H), 6.59-6.54 (d, 1H), 4.20 (s, 4H), 3.82 (s, 4H), 2.32-2.29 (d, 3H) ppm; ESI-MS obsd 339.1715, calcd 339.1703 [(M+H)⁺, M=C₂₀H₂₂N₂O₃].

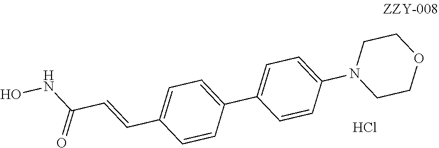 [SMILES: O=C(/C=C/c1ccc(-c2ccc(N3CCOCC3)cc2)cc1)NO ; Cl]

¹H NMR (300 MHz, MeOH-d4) δ8.07 (sbr, 1H), 7.91-7.88 (m, 2H), 7.82-7.79 (d, 1H), 7.76-7.74 (d, 2H), 7.71-7.62 (m, 4H), 6.60-6.55 (d, 1H), 4.17-4.16 (d, 4H), 3.82-3.76 (m, 4H) ppm; ESI-MS obsd 325.1549, calcd 325.1547 [(M+H)⁺, M=C₁₉H₂₀N₂O₃].

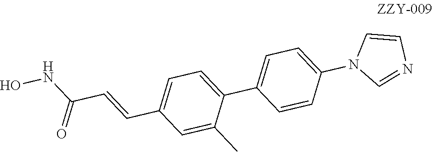 [SMILES: Cc1cc(/C=C/C(=O)NO)ccc1-c1ccc(-n2ccnc2)cc1]

¹H NMR (300 MHz, MeOH-d4) δ9.59 (sbr, 1H), 8.18 (s, 1H), 7.87-7.84 (d, 3H), 7.65-7.50 (m, 5H), 7.32-7.29 (d, 1H), 6.59-6.54 (d, 1H), 2.33 (s, 3H) ppm; ESI-MS obsd 320.1390, calcd 320.1394 [(M+H)⁺, M=C₁₉H₁₇N₃O₂].

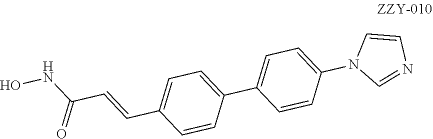 [SMILES: O=C(/C=C/c1ccc(-c2ccc(-n3ccnc3)cc2)cc1)NO]

¹H NMR (400 MHz, MeOH-d4) δ9.54 (sbr, 1H), 8.15 (s, 1H), 7.95-7.91 (m, 3H), 7.85-7.81 (m, 4H), 7.76-7.74 (d, 3H), 7.70-7.68 (m, 3H), 7.64-7.60 (d, 1H), 6.59-6.55 (d, 1H) ppm; ESI-MS obsd 306.1231, calcd 306.1237 [(M+H)⁺, M=C₁₈H₁₅N₃O₂].

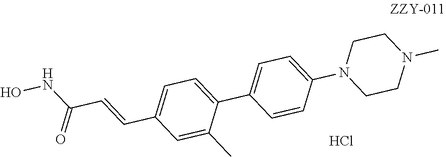 [SMILES: Cc1cc(/C=C/C(=O)NO)ccc1-c1ccc(N2CCN(C)CC2)cc1 ; Cl]

¹H NMR (300 MHz, MeOH-d4) δ7.65-7.60 (d, 1H), 7.48-7.42 (m, 2H), 7.29-7.11 (m, 2H), 7.16-7.13 (m, 2H), 7.02-6.92 (m, 1H), 6.57-6.52 (d, 1H), 3.96-3.64 (m, 4H), 3.33-3.19 (m, 4H), 3.01-3.00 (s, 3H), 2.28 (s, 3H) ppm; ESI-MS obsd 352.2020, calcd 352.2020 [(M+H)⁺, M=C₂₁H₂₅N₃O₂].

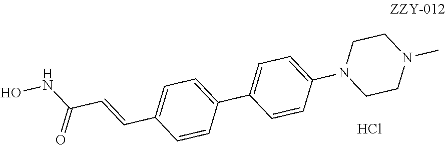 [SMILES: CN1CCN(c2ccc(-c3ccc(/C=C/C(=O)NO)cc3)cc2)CC1 ; Cl]

¹H NMR (300 MHz, MeOH-d4) δ7.70-7.59 (m, 6H), 7.43-7.38 (m, 1H), 7.30-7.23 (m, 1H), 7.14-7.06 (m, 1H), 6.57-6.49 (m, 1H), 3.97-3.91 (m, 2H), 3.68-3.61 (m, 2H), 3.34-3.29 (m, 2H), 3.21-3.13 (m, 2H), 3.01-3.00 (s, 3H) ppm; ESI-MS obsd 338.1865, calcd 338.1863 [(M+H)⁺, M=C₂₀H₂₃N₃O₂].

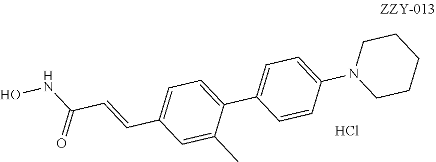 [SMILES: Cc1cc(/C=C/C(=O)NO)ccc1-c1ccc(N2CCCCC2)cc1 ; Cl]

¹H NMR (300 MHz, MeOH-d4) δ7.83-7.80 (d, 1H), 7.74-7.71 (m, 2H), 7.61-7.50 (m, 5H), 7.32-7.25 (m, 1H), 6.74-6.55 (m, 1H), 3.75-3.72 (m, 4H), 2.33-2.30 (s, 3H), 2.13-2.11 (m, 4H), 1.87 (s, 2H) ppm; ESI-MS obsd 337.1916, calcd 337.1911 [(M+H)⁺, M=C₂₁H₂₄N₂O₂].

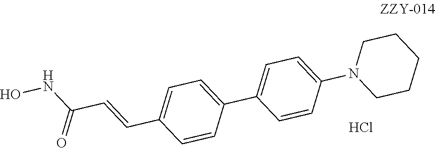 [SMILES: O=C(/C=C/c1ccc(-c2ccc(N3CCCCC3)cc2)cc1)NO ; Cl]

¹H NMR (300 MHz, MeOH-d4) δ7.88-7.71 (m, 9H), 6.73-6.59 (m, 1H), 3.71 (sbr, 4H), 2.12 (sbr, 4H); 1.86 (brs, 2H) ppm; ESI-MS obsd 323.1761, calcd 323.1754 [(M+H)⁺, M=C₂₀H₂₂N₂O₂].

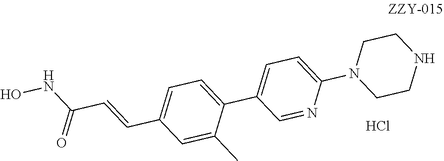 [SMILES: Cc1cc(/C=C/C(=O)NO)ccc1-c1ccc(N2CCNCC2)nc1 ; Cl]

¹H NMR (300 MHz, MeOH-d4) δ8.21-8.18 (d, 1H), 8.07 (s, 1H), 7.60-7.50 (d, 4H), 7.33-7.31 (d, 1H), 6.59-6.54 (d, 1H), 4.15 (sbr, 4H), 3.81 (sbr, 4H), 2.04 (s, 3H) ppm; ESI-MS obsd 339.1812, calcd 339.1816 [(M+H)⁺, M=C₁₉H₂₂N₄O₂].

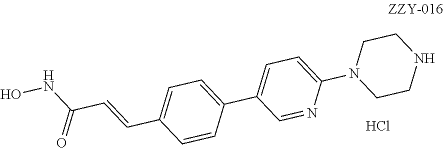 [SMILES: O=C(/C=C/c1ccc(-c2ccc(N3CCNCC3)nc2)cc1)NO ; Cl]

¹H NMR (300 MHz, MeOH-d4) δ8.50-8.46 (m, 1H), 8.38-8.37 (d, 1H), 7.77-7.69 (m, 4H), 7.63-7.59 (d, 1H), 7.58-7.56 (d, 1H), 6.59-6.54 (d, 1H), 4.11-4.07 (m, 4H), 3.55-3.51 (m, 4H) ppm; ESI-MS obsd 325.1656, calcd 325.1659 [(M+H)⁺, M=C₁₈H₂₀N₄O₂].

### Embodiment 6 In Vitro LSD1 Inhibitory Activity Detection

LSD1 activity was detected by using LSD1-HRP coupling reaction. The principle was shown in the following reaction formula: as can be seen from the reaction mechanism of substrate demethylation catalyzed by LSD1, by-product H₂O₂ was produced in this process, thus horseradish peroxidase (HRP) was used to catalyze the reaction of H₂O₂ with Amplex Red (a dye) to produce Resorufin (a highly fluorescent substance) and H₂O, and indirectly obtaining the inhibitory activity of the molecule to be tested on the LSD1 by detecting the fluorescence intensity of the product. During the reduction of H₂O₂ to H₂O, Amplex Red acts as an electron donor and oxidizes to Resorufin for detection of fluorescence. In this experiment, 535 nm was selected as the excitation wavelength and 595 nm as the emission wavelength for detection. Ampliflu™ Red, peroxidase, from horseradish required for the experiment was purchased from SIGMA; H3K4Me2 substrate peptide was purchased from GL Biochemical (Shanghai) Co., Ltd.

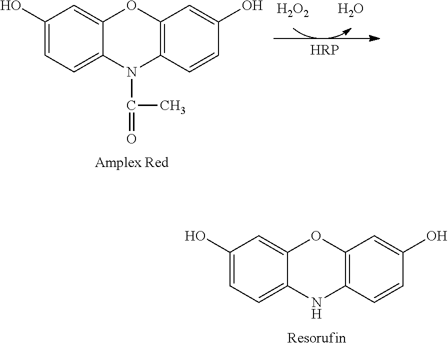

During the test, the recombinant human LSD1 protein with enzyme activity was first expressed in E. coli, and then the samples were prepared according to the 50 μl/well system: containing 10 μM HRP+50 μM Amplex Red+100 nM LSD1+buffer (25 mM Hepes, 250 mM NaCl, 5% Glycerol pH 7.5), added to the 96-well plate; the gradient diluted molecules to be tested were added to the 96-well plate, and the oscillatory reaction was carried out for 30 min at room temperature; the starting solution was prepared according to 50 μl/well system: containing 10 μM H3K4Me2 peptide+buffer, added to the 96-well plate, and after slight shaking, immediately detected by multi-function microplate enzyme marker (Ex: 535 nM; Em: 595 nM). The results are shown in Table 1 below: Table 1 shows the inhibitory activity of the compound of the present invention on blocking LSD1 enzyme activity in vitro.

TABLE 1

| Compounds | IC₅₀ (nM) | Compounds | IC₅₀ (nM) | Compounds | IC₅₀ (nM) | Compounds | IC₅₀ (nM) |
|---|---|---|---|---|---|---|---|
| ZZY-001 | 1549 | ZZY-005 | 5164 | ZZY-009 | 1405 | ZZY-013 | 1415 |
| ZZY-002 | 1320 | ZZY-006 | 2376 | ZZY-010 | 8264 | ZZY-014 | 1106 |
| ZZY-003 | 439.9 | ZZY-007 | 477 | ZZY-011 | 131.3 | ZZY-015 | 879.2 |
| ZZY-004 | 2487 | ZZY-008 | 715.6 | ZZY-012 | 16.09 | ZZY-016 | 991.1 |
| TCP | >30,000 | Vorinostat | >100,000 | | | | |

It can be seen from the IC₅₀ (nM) values of Table 1 that the ability of the compounds ZZY-001 to ZZY-016 to inhibit LSD1 was far superior to the LSD1 positive inhibitor TCP (tranylcypromine) and the HDAC positive inhibitor Vorinostat, so compounds ZZY-001 to ZZY-016 are highly effective inhibitors of LSD1.

### Embodiment 7 In Vitro HDAC1 Inhibitory Activity Detection

HDAC1 inhibitory activity was detected by using the FLUOR DE LYS® HDAC1 fluorometric drug discovery assay kit. The principle was shown in the following reaction formula: HDAC1 catalyzes the deacetylation of a substrate FLUOR DE LYS® Substrate (containing an acetylated side chain), the product can react with FLUOR DE LYS® Developer II to generate fluorescence (shown in the following reaction process), and the inhibitory activity of a molecule to be detected on HDAC1 can be indirectly obtained by detecting the fluorescence intensity of the product. In this experiment, 360 nm was selected as the excitation wavelength and 460 nm as the emission wavelength for detection. FLUOR DE LYS® HDAC1 fluorometric drug discovery assay kit required for experiments was purchased from Enzo Biochem Inc.

During the test, the sample was prepared according to the specific experimental conditions of blocking the HDAC1 enzyme activity of the compound of the present invention in vitro in accordance with Table 2 below. After slight oscillation at the end of the third step, the samples were detected immediately by multifunctional microplate enzyme marker (Ex: 360 nM; Em: 460 nM). The results are shown in Table 3 below: Table 3 shows the inhibitory activity of compounds of the present invention to block HDAC1 enzyme activity in vitro.

TABLE 2

| | | | | | The third step |
|---|---|---|---|---|---|
| | | | | The second step | (reacting for |
| | The first step (reacting for | | | (reacting for 60 min at | 45 min at 30° C. |
| | 30 min at 30° C. after oscillation) | | | 30° C. after oscillation) | after oscillation) |
| Sample | HDAC | HDAC1 | Inhibitor | Fluor de Lys ®-SIRT1 | Fluor de Lys ® |
| | Assay | (Dilution) | (5x) | Substrate (BML-KI177) | Developer II |
| | Buffer II | | | (2x) | |
| Test | 0 ul | 15 ul | 10 ul | 25 ul | 50 ul |
| Sample | | | | | |
| Positive | 10 ul | 15 ul | 0 ul | 25 ul | 50 ul |
| control | | | | | |
| Negative | 25 ul | 0 ul | 0 ul | 25 ul | 50 ul |
| control | | | | | |

TABLE 3

| Compounds | IC₅₀ (nM) | Compounds | IC₅₀ (nM) | Compounds | IC₅₀ (nM) | Compounds | IC₅₀ (nM) |
|---|---|---|---|---|---|---|---|
| ZZY-001 | 670.8 | ZZY-005 | 8943 | ZZY-009 | 2801 | ZZY-013 | 9643 |
| ZZY-002 | 71.57 | ZZY-006 | 5385 | ZZY-010 | 3522 | ZZY-014 | 9855 |
| ZZY-003 | 179.9 | ZZY-007 | 1112 | ZZY-011 | 434 | ZZY-015 | 262.4 |
| ZZY-004 | 12365 | ZZY-008 | 9358 | ZZY-012 | 942.4 | ZZY-016 | 451.2 |
| TCP | >100,000 | Vorinostat | 206.0 | | | | |

It can be seen from the IC₅₀ (nM) values of Table 3 that the ability of the compounds ZZY-001 to ZZY-016 to inhibit HDAC1 was similar to HDAC positive inhibitor vorinostat, and was far superior to the LSD1 positive inhibitor TCP, so compounds ZZY-001 to ZZY-016 are highly effective inhibitors of HDAC1.

It can be seen by combining the IC₅₀ (nM) values of Tables 1 and Table 3: compounds of the embodiments ZZY-001 to ZZY-016 can simultaneously inhibit the activity of LSD1 and HDAC1, and are bifunctional inhibitors of LSD1 and HDAC.

### Embodiment 8 In Vitro Tumor Cell Inhibitory Activity Detection of the Compound of the Present Invention

MTT colorimetric method was used to detect the inhibitory activity of a series of compounds (i.e. ZZY-001 to ZZY-016, a total of 16 kinds) of the embodiment of the present invention on the proliferation of tumor cells in vitro. The full name of MTT is 3-(4,5-dimethyl-2-thiazolyl)-2,5-diphenyl-2-H-tetrazolium romide, which is a yellow dye also known as thiazolyl blue. The detection principle was as follows: succinate dehydrogenase in the mitochondria of living cells reduced MTT to water-insoluble blue-violet crystalline Formazan, but dead cells do not have this function. DMSO can dissolve Formazan deposited in cells and the absorbance value can be measured with a enzyme marker at a wavelength of 490 nm. In a certain range of cell number, the production of Formazan is proportional to the number of cells, so the number of living cells can be inferred according to the measured OD value.

In the experiments of this embodiment, the selected cell lines are human breast cancer cell line MDA-MB-231 and BT-474, human colon adenocarcinoma cell HCT116, mouse colon cancer cell CT26.WT, mouse breast cancer cell 4T1. During the test, the cells in logarithmic phase and in good condition were digested with 0.25% trypsin digestion solution to make the adherent cells fall off, and the cell suspension (suspension cells without trypsin digestion) was made by counting 2-4×10⁴ cells/ml. The cell suspension was inoculated on a 96-well plate at 100 μl/well and cultured in a constant temperature CO₂ incubator for 24 h. Then the tested drugs were added, 100 μl/well, and cultured for 72 h. MTT was added to a 96-well plate, 20 μl/well, and reacted for 4 h in the incubator. Absorb the supernatant, add DMSO, 150 μl/well and shake for 10 min on the plate shaker. The optical density (OD value) of each well was measured by enzyme marker at the wavelength of 490 nm. The results are shown in Table 4 below: Table 4 shows that compounds of several embodiments, ZZY-001, ZZY-002, ZZY-003, ZZY-011 and ZZY-012 have significant inhibitory activities on several human tumor cells HCT116, MDA-MB-231, BT-474, and mouse tumor cells CT26.WT and 4T1, wherein, the positive inhibitor of LSD1, TCP (anti-amphetamine), and the positive inhibitor of HDAC, Vorinostat, served as control.

TABLE 4

| | IC₅₀ (nM) | | | | |
|---|---|---|---|---|---|
| Compounds | HCT116 | MDA-MB-231 | BT-474 | CT26.WT | 4T1 |
| ZZY001 | 1540 | 2316 | 17826 | 2205 | 3869 |
| ZZY002 | 1288 | 1368 | 7564 | 31153 | 5607 |
| ZZY003 | 265.4 | 835.6 | 1861 | 8759 | 2379 |
| ZZY011 | 728.2 | 2037 | 5904 | 1047 | 2597 |
| ZZY012 | 468.7 | 1139 | 4214 | 736.7 | 2317 |
| TCP | >10,000 | >10,000 | >10,000 | >10,000 | >10,000 |
| Vorinostat | 1445 | 1580 | 3658 | 4355 | 2693 |

### Embodiment 9 Cell Proliferation of 50 Cell Lines by the Inventive Compound ZZY-003

The effect of the present invention compound ZZY-003 on the cell proliferation of 50 cell lines was evaluated by CellTiter-Glo (CTG) method, and the 50% inhibitory concentration was calculated by detecting the cell viability after treatment with different drug concentrations. The cells were resuscitated and cultured in their respective culture media. The cells in logarithmic growth phase were harvested and the cells were counted by cell counter. The cell viability was detected by Trypan blue exclusion method to ensure that the cell line viability was more than 96%. The cell concentration was adjusted by diluting with the culture medium, and 90 μL cell suspension was added to the 96-well cell plate (including the cell control T0 on the day of drug treatment) to make the cell density reach the specified concentration. The cells in the 96-well plate were cultured overnight at 37° C., 5% CO₂ and 95% humidity. 10 μL culture medium was added to the control cell culture plate. CellTiter-Glo reagent and cell culture plate were placed at room temperature to balance for 30 minutes. The same volume of CellTiter-Glo reagent was added to each well. Vibrate on the orbital shaker for 2 min to make the cells fully lytic. The cell culture plate was placed at room temperature to balance for 10 minutes. Chemiluminescence values were read with EnVision. The storage solution was formed by dissolving the tested compound with the corresponding solvent and diluted in gradient to obtain 10 times working concentration solution, and 10 times solution of positive drug was also prepared. 10 μL drug solution was added to each well in the 96-well plate in which the cells were inoculated, and each cell concentration was provided with three duplicate wells. The highest concentration of the tested compound was 10/50 μM, 9 concentrations and 3.16 times dilution. The cells in the 96-well plate added with drug were cultured at 37° C., 5% CO₂ and 95% humidity for 72 hours. CellTiter-Glo reagent and drug-treated cell culture plate were placed at room temperature to balance for 30 minutes. The same volume of CellTiter-Glo reagent was added to each well. Vibrate on the orbital shaker for 2 minutes to make the cells fully lytic. The cell culture plate was placed at room temperature to balance for 10 minutes. Chemiluminescence values were read with EnVision.

Data processing: GraphPad Prism 5.0 software was used to analyze the data, nonlinear S-curve regression was used to fit the data to get the dose-effect curve, and the IC₅₀ value was calculated. The specific data are shown in Table 5, and Table 5 shows the IC₅₀ value and the highest concentration inhibition rate of compound ZZY-003 on 50 cell lines.

Cell survival rate (%)=(Lum drug to be detected−Lum culture medium control)/(Lum cell control−Lum culture medium control)×100%;

Lum cell control−Lum culture medium control was set to 100%, LumMedium control value was set to 0%;

amplification fold=(5th day LumNone treated—LumMedium control)/(2nd day LumNone treated—LumMedium control).

TABLE 5

| Cell | | Absolute IC₅₀(μM) | | % inhibition at top conc. | |
|---|---|---|---|---|---|
| No. | Cell lines | ZZY-003 | Cisplatin | ZZY-003 | Cisplatin |
| 1 | LP-1 | 0.26 | 8.68 | 98.94% | 99.89% |
| 2 | MDA-MB-231 | 1.81 | 20.61 | 92.06% | 85.71% |
| 3 | NCI-H1299 | 3.40 | 3.69 | 99.94% | 98.91% |
| 4 | AN3 CA | 1.27 | 4.43 | 99.92% | 99.96% |
| 5 | SK-MEL-28 | 1.24 | 8.61 | 99.89% | 99.34% |
| 6 | SNU-761 | 9.32 | 4.52 | 99.35% | 99.92% |
| 7 | MDA-MB-453 | 0.19 | 12.92 | 99.93% | 95.15% |
| 8 | 143B | 2.25 | 1.15 | 99.97% | 99.94% |
| 9 | PC-3 | 1.38 | 5.34 | 96.56% | 85.52% |
| 10 | MCF7 | 1.61 | 21.96 | 96.26% | 90.30% |
| 11 | SNU-5 | 1.64 | 2.45 | 99.83% | 97.87% |
| 12 | A549 | 2.21 | 7.89 | 95.22% | 96.72% |
| 13 | PANC-1 | 5.84 | 8.67 | 96.79% | 94.45% |
| 14 | SW480 | 1.74 | 2.69 | 99.24% | 94.12% |
| 15 | EBC-1 | 1.15 | 17.77 | 92.30% | 85.11% |
| 16 | 5637 | 0.82 | 2.60 | 99.95% | 99.05% |
| 17 | MOLM-13 | 0.21 | 0.77 | 99.99% | 99.99% |
| 18 | NCI-H82 | 0.37 | 4.00 | 99.94% | 99.63% |
| 19 | SK-OV-3 | 1.59 | 5.17 | 99.68% | 89.16% |
| 20 | SK-N-FI | 3.17 | 10.18 | 99.95% | 99.81% |
| 21 | B16-F10 | 1.03 | 4.53 | 99.98% | 99.97% |
| 22 | MIA PaCa-2 | 0.48 | 6.05 | 99.94% | 99.18% |
| 23 | SU-DHL-6 | 0.30 | 5.65 | 99.91% | 99.95% |
| 24 | U266B1 | 0.64 | 14.31 | 98.03% | 98.85% |
| 25 | H69AR | 4.36 | 5.65 | 96.23% | 94.93% |
| 26 | DLD-1 | 5.13 | 5.30 | 97.98% | 98.93% |
| 27 | CNE-2Z | 1.52 | 1.18 | 99.84% | 96.44% |
| 28 | HeLa | 1.40 | 0.37 | 98.88% | 99.45% |
| 29 | K-562 | 0.46 | 6.07 | 99.85% | 97.45% |
| 30 | Jurkat clone E6-1 | 0.48 | 0.84 | 99.61% | 99.96% |
| 31 | U-937 | 0.46 | 2.19 | 99.99% | 99.97% |
| 32 | HL-60 | 0.31 | 1.56 | 99.93% | 99.99% |
| 33 | HuCCT1 | 1.35 | 11.28 | 99.88% | 89.54% |
| 34 | JVM-3 | 0.35 | 0.70 | 99.48% | 99.71% |
| 35 | Ramos | 0.32 | 1.78 | 99.82% | 99.98% |
| 36 | OE19 | 0.78 | 25.81 | 98.95% | 62.99% |
| 37 | BT474 | 0.51 | 66.09 | 99.86% | 67.02% |
| 38 | Hep G2 | 0.84 | 5.86 | 99.98% | 90.70% |
| 39 | T84 | 9.95 | 5.05 | 98.70% | 87.03% |
| 40 | AMO-1 | 1.52 | 0.95 | 99.95% | 99.99% |
| 41 | HCT-116 | 0.43 | 3.15 | 99.94% | 95.72% |
| 42 | AZ-521 | 0.68 | 2.89 | 99.95% | 99.92% |
| 43 | NCI-H1417 | 0.33 | 4.40 | 97.14% | 81.11% |
| 44 | SF268 | 1.09 | 2.87 | 99.82% | 97.98% |
| 45 | UO.31 | 4.56 | 4.23 | 99.92% | 97.89% |
| 46 | 786-O | 3.91 | 2.11 | 99.96% | 97.88% |
| 47 | LN-229 | 1.57 | 11.82 | 99.97% | 97.32% |
| 48 | HT-1376 | 2.28 | 4.10 | 99.41% | 88.74% |
| 49 | OPM2 | 0.35 | 3.67 | 98.99% | 99.97% |
| 50 | MCF 10A | 1.49 | 13.20 | 97.73% | 99.51% |

The data from Table 5 shows: the compound ZZY-003 of the present invention has significant inhibitory activity on 50 kinds of tumor cells, and the inhibitory activity on most of the detected tumor cells is stronger than that of the control chemotherapy drug Cisplatin, which fully shows that the compound of the present invention has broad-spectrum anti-tumor activity. While the foregoing is directed to the preferred examples of the present invention, it is not intended to limit the present invention to the precise form disclosed, any modification, equivalent substitution and improvement made within the spirit and principle of the present application should be included in the protection scope of the present application.

What is claimed is:

1. A compound, having a general structural formula as shown in Formula I:
X-AB-Y Formula I
In the above Formula I,
X is selected from any one of —CONHZ, —CH═CH—CO₂H, —CH═CH—CONHZ, wherein Z is selected from any one of substituted or unsubstituted C₁-C₁₂ alkyl, substituted or unsubstituted aryl, and hydroxyl;
Y═—NR₁R₂, wherein NR₁R₂ is a substituted or unsubstituted 3- to 9-membered nitrogen-containing heterocycloalkyl;
A and B are each independently selected from substituted or unsubstituted phenylene, substituted or unsubstituted azaphenylene.

2. The compound of claim 1, wherein in the Formula I, A and B are each independently selected from:
any one of 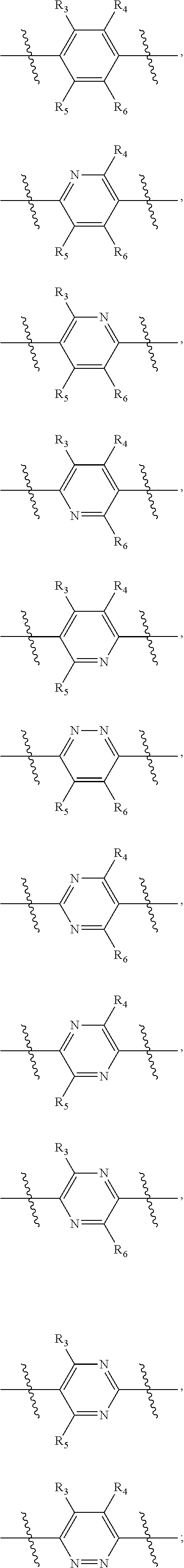
Wherein R₃, R₄, R₅ and R₆ are each independently selected from any one of substituted or unsubstituted C₁-C₁₂ alkyl, substituted or unsubstituted C₁-C₁₂ heteroalkyl, substituted or unsubstituted C₃-C₁₂ cycloalkyl, substituted or unsubstituted C₃-C₁₂ heterocycloalkyl, substituted or unsubstituted C₂-C₁₂ alkenyl, substituted or unsubstituted C₂-C₁₂ heteroalkenyl, substituted or unsubstituted C₃-C₁₂ cycloalkenyl, substituted or unsubstituted C₃-C₁₂ heterocycloalkenyl, substituted or unsubstituted C₂-C₁₂ alkynyl, substituted or unsubstituted C₂-C₁₂ heteroalkynyl, substituted or unsubstituted C₃-C₁₂ cycloalkynyl, substituted or unsubstituted C₃-C₁₂ heterocycloalkynyl, substituted or unsubstituted aryl, substituted or unsubstituted heteroaryl, aryl (C₁-C₁₂) alkyl, heteroaryl (C₁-C₁₂) alkyl, C₂-C₁₂ alkenyl (C₁-C₁₂) alkyl, C₂-C₁₂ alkynyl (C₁-C₁₂) alkyl, hydroxyl, amino, nitro, sulfo, halogen and hydrogen atom.

3. The compound of claim 2, wherein in the Formula I, R₃, R₄, R₅ and R₆ are each independently selected from any one of substituted or unsubstituted C₁-C₆ alkyl, substituted or unsubstituted C₁-C₆ heteroalkyl, substituted or unsubstituted C₃-C₆ cycloalkyl, substituted or unsubstituted C₃-C₆ heterocycloalkyl, substituted or unsubstituted C₂-C₆ alkenyl, substituted or unsubstituted C₂-C₆ heteroalkenyl, substituted or unsubstituted C₃-C₆ cycloalkenyl, substituted or unsubstituted C₃-C₆ heterocycloalkenyl, substituted or unsubstituted C₂-C₆ alkynyl, substituted or unsubstituted C₂-C₆ heteroalkynyl, substituted or unsubstituted C₃-C₆ cycloalkynyl, substituted or unsubstituted C₃-C₆ heterocycloalkynyl, substituted or unsubstituted aryl, substituted or unsubstituted heteroaryl, aryl (C₁-C₆) alkyl, heteroaryl (C₁-C₆) alkyl, C₂-C₆ alkenyl (C₁-C₆) alkyl, C₂-C₆ alkynyl (C₁-C₆) alkyl.

4. The compound of claim 2, wherein in the Formula I, a structure of -AB— is selected from:
any one of 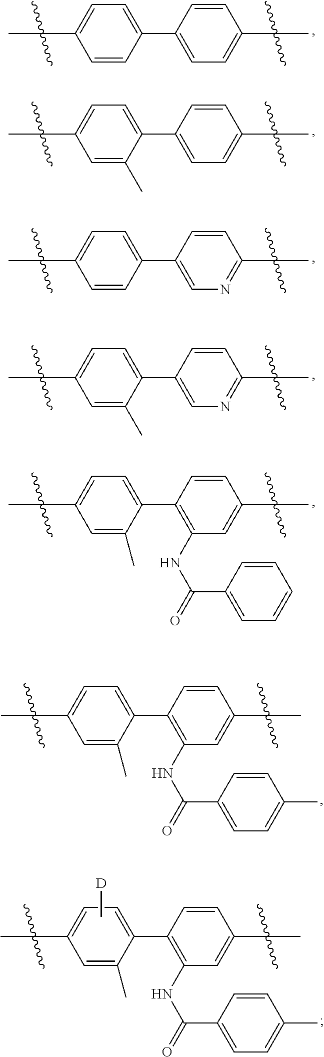
wherein D is halogen.

5. The compound of claim 1, wherein in the Formula I, Z is substituted or unsubstituted C₁-C₄ alkyl or hydroxy.

6. The compound of claim 1, wherein in the Formula I, a structure of Z is 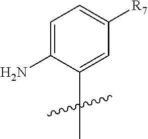
or hydroxyl; wherein R₇ is selected from any one of substituted or unsubstituted C₁-C₁₂ alkyl, substituted or unsubstituted C₁-C₁₂ heteroalkyl, substituted or unsubstituted C₃-C₁₂ cycloalkyl, substituted or unsubstituted C₃-C₁₂ heterocycloalkyl, substituted or unsubstituted C₂-C₁₂ alkenyl, substituted or unsubstituted C₂-C₁₂ heteroalkenyl, substituted or unsubstituted C₃-C₁₂ cycloalkenyl, substituted or unsubstituted C₃-C₁₂ heterocycloalkenyl, substituted or unsubstituted C₂-C₁₂ alkynyl, substituted or unsubstituted C₂-C₁₂ heteroalkynyl, substituted or unsubstituted C₃-C₁₂ cycloalkynyl, substituted or unsubstituted C₃-C₁₂ heterocycloalkynyl, substituted or unsubstituted aryl, substituted or unsubstituted heteroaryl, aryl (C₁-C₁₂) alkyl, heteroaryl (C₁-C₁₂) alkyl, C₂-C₁₂ alkenyl (C₁-C₁₂) alkyl, C₂-C₁₂ alkynyl (C₁-C₁₂) alkyl, hydroxyl, amino, nitro, sulfo, halogen and hydrogen atom.

7. The compound of claim 1, wherein in the Formula I, Y is a substituted or unsubstituted 5- or 6-membered nitrogen-containing heterocycloalkyl.

8. The compound of claim 7, wherein Y is selected from:
any one of

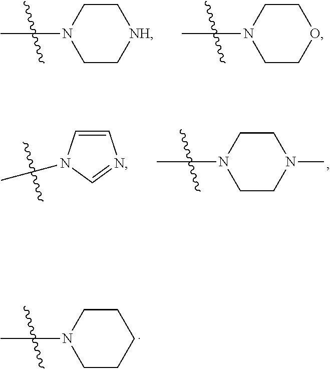

9. The compound of claim 1, wherein the compound is selected from any one of

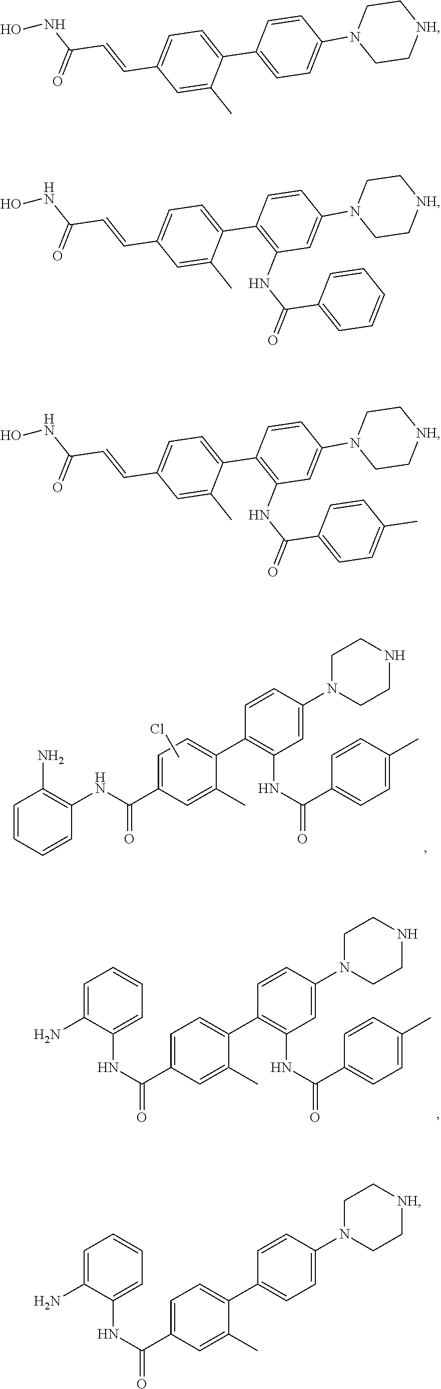

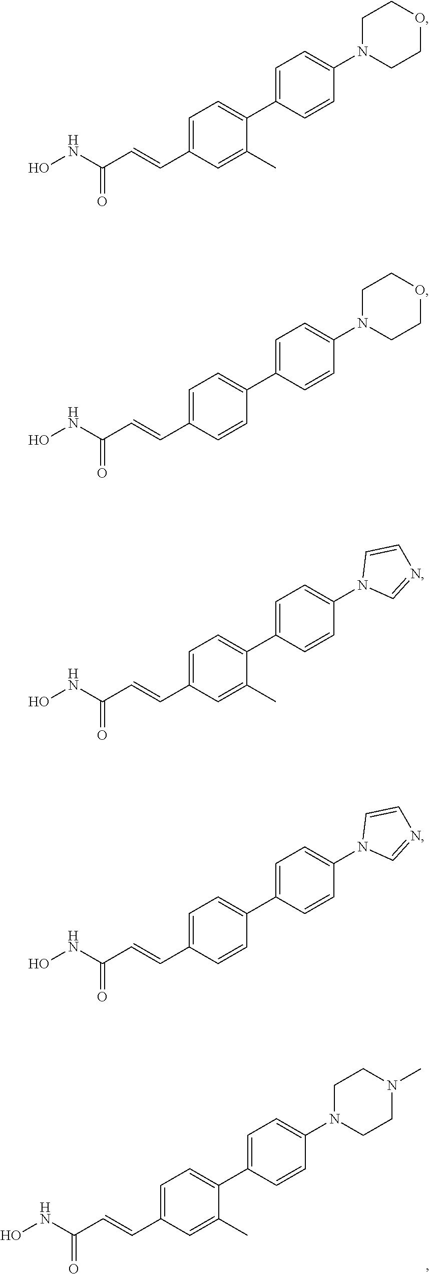 [SMILES: Cc1cc(/C=C/C(=O)NO)ccc1-c1ccc(N2CCOCC2)cc1 ; O=C(/C=C/c1ccc(-c2ccc(N3CCOCC3)cc2)cc1)NO ; Cc1cc(/C=C/C(=O)NO)ccc1-c1ccc(-n2ccnc2)cc1 ; O=C(/C=C/c1ccc(-c2ccc(-n3ccnc3)cc2)cc1)NO ; Cc1cc(/C=C/C(=O)NO)ccc1-c1ccc(N2CCN(C)CC2)cc1]

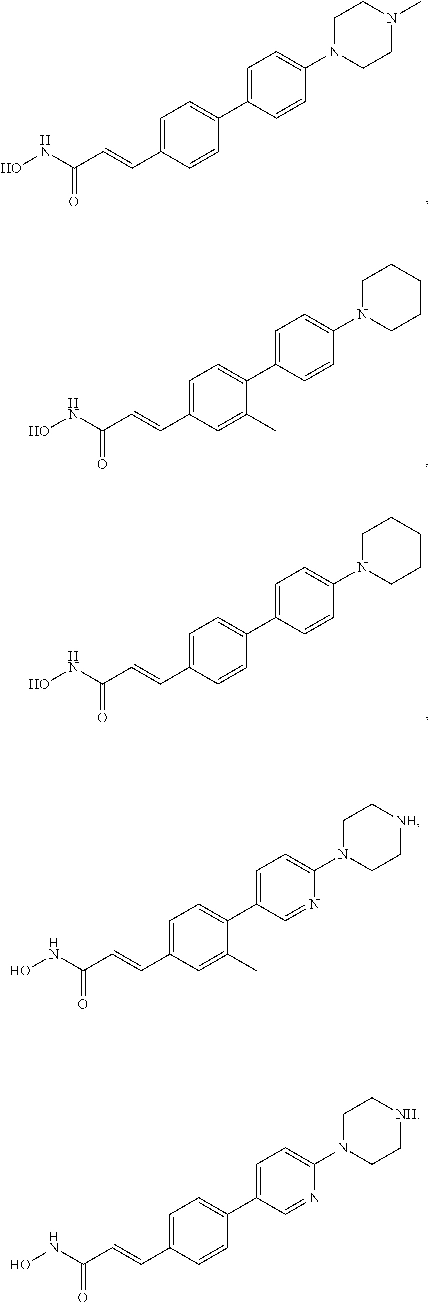 [SMILES: CN1CCN(c2ccc(-c3ccc(/C=C/C(=O)NO)cc3)cc2)CC1 ; Cc1cc(/C=C/C(=O)NO)ccc1-c1ccc(N2CCCCC2)cc1 ; O=C(/C=C/c1ccc(-c2ccc(N3CCCCC3)cc2)cc1)NO ; Cc1cc(/C=C/C(=O)NO)ccc1-c1ccc(N2CCNCC2)nc1 ; O=C(/C=C/c1ccc(-c2ccc(N3CCNCC3)nc2)cc1)NO]

10. A pharmaceutical composition comprising the compound of claim 1 and a pharmaceutically acceptable carrier.

11. A pharmaceutical composition comprising the compound of claim 2 and a pharmaceutically acceptable carrier.

12. A pharmaceutical composition comprising the compound of claim 3 and a pharmaceutically acceptable carrier.

13. A pharmaceutical composition comprising the compound of claim 4 and a pharmaceutically acceptable carrier.

14. The pharmaceutical composition of claim 10, wherein the pharmaceutical composition further comprises an anticancer drug.

15. The pharmaceutical composition of claim 14, wherein the anticancer drug is an LSD1 inhibitor and/or an HDAC inhibitor.

16. Use of the compound of claim 1 or a pharmaceutically acceptable salt thereof in preparation of a drug for the treatment and/or prevention of a tumor or cancer associated with LSD1 and/or HDAC.

17. Use of the compound of claim 2 or a pharmaceutically acceptable salt thereof in preparation of a drug for the treatment and/or prevention of a tumor or cancer associated with LSD1 and/or HDAC.

18. Use of the compound of claim 3 or a pharmaceutically acceptable salt thereof in preparation of a drug for the treatment and/or prevention of a tumor or cancer associated with LSD1 and/or HDAC.

19. Use of the compound of claim 4 or a pharmaceutically acceptable salt thereof in preparation of a drug for the treatment and/or prevention of a tumor or cancer associated with

20. The use of claim 16, wherein the tumor or cancer is selected from: at least one of brain cancer, glioblastoma, leukemia, Bristlecone syndrome, Cowden's disease, cerebellar dysplastic gangliocytoma, breast cancer, inflammatory breast cancer, Wilm's tumor, Ewing's sarcoma, rhabdomyosarcoma, ependymoma, medulloblastoma, colon cancer, head and neck cancer, kidney cancer, lung cancer, liver cancer, melanoma, ovarian cancer, pancreatic cancer, prostate cancer, sarcoma, osteosarcoma, giant cell tumor of the bone and thyroid.
